(19) United States
(12) Patent Application Publication
(10) Pub. No.: US 2011/0300554 A1
(43) Pub. Date: Dec. 8, 2011

(54) EXPONENTIAL ISOTHERMAL SELF-SUSTAINED REPLICATION OF AN RNA ENZYME

(75) Inventors: Gerald F. Joyce, Rancho Santa Fe, CA (US); Bianca J. Lam, Encinitas, CA (US)

(73) Assignee: The scripps Research Institute

(21) Appl. No.: 13/138,057

(22) PCT Filed: Dec. 31, 2009
(86) PCT No.: PCT/US2009/006762

§ 371 Date: Aug. 17, 2011

(51) Int. Cl. C12Q 1/68; C07H 21/02; G01N 33/53; C07H 21/00; G01N 33/50; G01N 33/68

(52) U.S. Cl. 435/6.19; 435/6.1; 436/94; 436/501; 536/23.1

(57) ABSTRACT

This invention provides nucleic acid molecules that catalyze their own replication and undergo exponential amplification at a constant temperature and in the absence of proteins or other biological components, such as those employed in other amplification reactions, e.g., proteins including DNA or RNA polymerase and a method of detect a selected molecule using said nucleic acid.

### CROSS-REFERENCE TO RELATED APPLICATIONS

This application claims the benefit of the filing date of U.S. application Ser. No. 61/142,290, filed Jan. 2, 2009 and U.S. application Ser. No. 61/143,111, filed Jan. 7, 2009, the disclosures of which are incorporated by reference herein.

### STATEMENT OF GOVERNMENT RIGHTS

The invention was made with a grant from the Government of the United States of America (grant GM065130 from the National Institutes of Health). The Government may have certain rights to the invention.

### BACKGROUND

A longstanding research goal has been to devise a non-biological system that undergoes replication in a self-sustained manner, brought about by enzymatic machinery which is part of the system being replicated. Most commonly, this has involved reactions of the form A+B→T, where A and B are two substrates that bind to a template T and become joined to form a new copy of T. One way to realize the goal of a non-biological system that undergoes replication in a self-sustained manner, inspired by the notion of primitive RNA-based life, would be for an RNA enzyme to catalyze the replication of RNA molecules, including the RNA enzyme itself (Crick, 1968; Szostak et al., 2001; Joyce, 2002; Orgel et al., 2004).

More complicated chemical self-replication systems have been devised that involve two templates that direct each other's synthesis: a template T directs the joining of A′ and B′ to form T′, while a template T′ directs the joining of A and B to form T (Sievers and von Kiedrowski, 1994; Lee et al. 1997). Such systems more closely resemble biological self-replication, which involves the synthesis of cross-complementary (rather than self-complementary) nucleic acid templates. Unlike biological systems, however, these chemical systems do not entail a replicative machinery. Once the substrates are bound at adjacent positions on the template, they become joined through a favorable reaction between reactive groups at their opposed ends. There also is an example of a cross-catalytic amplification system involving two deoxyribozymes, each of which catalyzes a cleavage reaction, rather than a joining reaction, although it is not self-replicating (Levy and Ellington, 2003).

It is difficult to design a self-replicating system that involves a separate replicative machinery because the machinery must also be copied and provided to each of the “progeny.” One approach toward this goal has been to devise self-replicating molecules that function as both template and machinery. For example, a self-replicating ribozyme was developed that binds two RNA substrates through Watson-Crick pairing and catalyzes their joining to form another copy of the ribozyme (Paul and Joyce, 2002). The copies behave in a similar manner, resulting in autocatalytic behavior. More recently, Kim and Joyce (2004) reported a cross-catalytic system with two ribozymes.

### SUMMARY OF THE INVENTION

The invention provides nucleic acid molecules, e.g., RNA molecules, that catalyze their own replication (self-replicating) (nucleic acid enzyme molecules) and undergo exponential amplification at a constant temperature (isothermal conditions) and in the absence of proteins or other biological components, such as those employed in other amplification reactions, e.g., proteins including DNA or RNA polymerases. Thus, a nucleic acid enzyme molecule of the invention is one that under appropriate conditions, e.g., constant temperatures of about 15° C. to about 55° C., provides for an increase in the copy number of its complement. In one embodiment, a self-replicating nucleic acid molecule of the invention provides for an exponential increase in the copy number. In one embodiment, the self-replicating nucleic acid molecule is cross-catalytic. In one embodiment, the self-replicating nucleic acid molecule is a ligase, such as one that joins two or more nucleic acid substrates. In one embodiment, the self-replicating nucleic acid molecule is a ligase that joins substrates where the 3′ end of one of the substrates includes a hydroxyl group and the 5′ end of the other substrate has a nucleotide triphosphate, e.g., pppG. In one embodiment, the self-replicating nucleic acid molecule is a ligase that joins substrates where the 3′ end of one of the substrates includes an amine group and the 5′ end of the other substrate has a nucleotide triphosphate. In one embodiment, the self-replicating nucleic acid molecule is a ligase that joins substrates where the 3′ end of one of the substrates includes a hydroxyl group and the 5′ end of the other substrate includes an alkyl phosphate group. In one embodiment, the self-replicating nucleic acid molecule of the invention is a RNA molecule. In one embodiment, the self-replicating nucleic acid molecule of the invention or its progeny, or substrates thereof, include modified nucleotides which are nuclease resistant, e.g., 2′ amino-2′-deoxypyrimidines or 2′-O-methyl purines (see, e.g., Fitzwater et al., 1996; Ciesiolka et al., 1996; and Lin et al., 1994, the disclosures of which are incorporated by reference herein) which optionally do not substantially reduce the activity of the molecule.

The catalytic activity of these self-replicating nucleic acid molecules, such as self-replicating RNA molecules, may be made dependent on the presence of a target ligand by linking the catalytic portion of the molecule to a ligand binding domain (aptamer), thereby providing a self-replicating aptazyme. In one embodiment, the catalytic activity of a cross-catalytic nucleic acid molecule, such as a cross-catalytic RNA molecule, may be made dependent on the presence of a target ligand by linking the catalytic portion of the molecule to a ligand binding domain, thereby providing an autocatalytic aptazyme. In one embodiment, exponential amplification of at least one of a pair of cross-catalytic nucleic acid molecules occurs in the presence, but not the absence, of the ligand. This provides a powerful means for detecting an analyte, such as a small molecule or protein in a sample. In one embodiment, the exponential growth rate of the self-replicating nucleic acid molecule depends on the concentration of the analyte, enabling one to determine the concentration of an analyte in an unknown sample. In one embodiment, a self-replicating aptazyme senses the ligand and after that produces a product template that no longer includes the ligand binding domain, and that template is exponentially amplified in a ligand independent manner. Such a system may also be employed, for instance, to control gene expression and in molecular computation.

In one embodiment, as described herein below, a cross-catalytic system, involving two RNA enzymes that catalyze each other's synthesis from a total of four component substrates and provide for self-sustained exponential amplification in the absence of proteins or other biological materials, was prepared. The system provides for amplification with a doubling time of about one hour, which can be continued indefinitely. Populations of various cross-replicating enzymes were constructed and allowed to compete for a common pool of substrates, in which the population underwent overall amplification of >10²⁵-fold, during which recombinant replicators arose and grew to dominate the population. These replicating RNA enzymes can serve as an experimental model of a genetic system. Many such model systems could be constructed, allowing different selective outcomes to be related to the underlying properties of the genetic system.

Thus, the invention provides a method to alter one or more properties of nucleic acid enzyme molecules such as RNA enzymes including cross-catalytic RNA enzymes. The method includes mutating one or more of: at least one substrate for a nucleic acid enzyme molecule, e.g., a ribozyme, the ribozyme, e.g., a first cross-catalytic RNA enzyme of a pair, both the substrate and the ribozyme, to produce a mutagenized population. Then progeny of the mutagenized population(s) are selected for a desired property. In one embodiment, the invention provides a method to enhance the catalytic properties of cross-catalytic RNA enzymes. The method includes mutating at least one of two substrates for a first cross-catalytic RNA enzyme of a pair and/or the first cross-catalytic RNA enzyme, to produce a first mutagenized population and/or mutating at least one of two substrates for a second cross-catalytic RNA enzyme of the pair and/or the second cross-catalytic RNA enzyme, to produce a second mutagenized population. Progeny of the first and/or second populations are selected, e.g., to have shorter reaction times, for instance, when competition for substrate is high (substrate concentration is low), relative to the first or second cross-catalytic RNA enzyme, and isolated. In one embodiment, the selected progeny comprise a G or a U at a position corresponding to the 3′ nucleotide, or a position within about 5 to about 20 nucleotides of the 3′ nucleotide, relative to one of the substrates that is not present in that position in the first or second self-replicating nucleic acid molecule. In one embodiment, the selected progeny comprise a G or a U at a position corresponding to the 3′ nucleotide and at a position within about 5 to about 20 nucleotides of the 3′ nucleotide of one of the substrates that are not present in that position in the first or second self-replicating nucleic acid molecule. In one embodiment, the 5′ end of one of the substrates is covalently linked to the first or second self-replicating nucleic acid molecule. In one embodiment, the 5′ phosphate containing substrate is covalently linked to the first or second self-replicating nucleic acid molecule prior to mutating. The mutagenesis may include random mutagenesis, mutagenic PCR, recombination mutagenesis, site directed mutagenesis, or any combination thereof.

As also described herein, a system is provided that combines the sensitivity of exponential amplification with the specificity that results from dynamically sensing a ligand throughout the course of amplification. Ligand dependent exponential amplification provides a powerful means for detecting any ligand that can be recognized by a nucleic acid aptamer. In one embodiment, the aptamer has pre-defined equilibrium (K_{d}), rate (k_{off}, kₒₙ) constants and thermodynamic (ΔH, ΔS) parameters of aptamer-target interaction. It does so in a quantitative manner, allowing one to determine the concentration of ligand in an unknown sample. The method is analogous to PCR-based detection of nucleic acids, but can be generalized to a wide variety of targets, including small molecules and proteins that are relevant to, for instance, medical diagnostics, screening assays, monitoring levels of therapeutic molecules in physiological samples, and environmental monitoring, or any chemically distinguishable molecule, such as a surface or particular architecture. Unlike PCR-based methods, however, the method of amplification of the invention does not require temperature cycling and does not depend on proteins or any other biological materials other than the ligand, which may be any molecule. Moreover, the method may be co-dependent on two different ligands, which allows one to analyze two different molecules or two different epitopes of the same molecule. The latter may be advantageous in achieving enhanced specificity for complex target molecules.

The invention provides a method to detect a selected molecule in a sample. The method includes contacting a sample suspected of having the selected molecule, a pair of cross-catalytic nucleic acid ligase molecules, wherein at least one of the pair comprises a ligand binding domain for the selected molecule, and substrates for each of the pair, under conditions that result in selected molecule-dependent ligation of substrates for the ligand binding domain containing nucleic acid molecule which yields product template and subsequent exponential amplification of that template. The presence or amount of the amplified template is detected or determined, thereby detecting or determining the presence or amount of the selected molecule in the sample.

In one embodiment, concentrations of about 1 to 100 μM of the selected molecule in the sample are detected or determined. In one embodiment, herein concentrations of about 1 to 100 mM of the selected molecule in the sample are detected or determined.

The invention further provides a composition comprising a pair of cross-catalytic RNA enzymes, wherein at least one of the pair comprises a ligand binding domain. In one embodiment, the RNA enzymes are ligases.

The system is thus useful in many applications, e.g., to detect structures or analytes found in physiological samples, e.g., drugs or metabolites, biological samples, including whole cells or organisms, proteins, isoforms of proteins, modified molecules such as phosphorylated molecules, and the like, environmental samples, such as mercury or dioxin detection, or other biosensing applications, for instance, biodefense, e.g., to detect spores of Bacillus anthracis. The detection may be conducted in a laboratory or in the field, as temperature cycling is not required for amplification. Moreover, the rate of amplification is a measure of the target ligand.

It would be straightforward to perform ligand dependent exponential amplification in a multiplex format. The two enzymes of a cross-replicating pair recognize each other through regions of Watson-Crick pairing, and these can be varied in sequence so long as they are complementary. Multiple cross-replicating pairs have been constructed that amplify faithfully when placed in a common reaction mixture, and each of these could be fitted with a different aptamer domain, so that many ligands could be assayed simultaneously. The system may thus be readily multiplexed by employing two or more cross-replicating pairs, e.g., with specificity for a plurality of different molecules, that amplify faithfully in a common reaction mixture. A means to distinguish the various ligated products may be based on, for example, unique sequence tags or distinct fluorescent signals. Such methods are well known in molecular detection.

### BRIEF DESCRIPTION OF THE FIGURES

FIG. 1. Cross-replicating RNA enzymes. (A) The enzyme E′ (gray) catalyzes ligation of substrates A and B (black) to form the enzyme E, while E catalyzes ligation of A′ and B′ to form E′. The two enzymes dissociate to provide copies that can catalyze another reaction. (B) Sequence and secondary structure of the complex formed between the enzyme and its two substrates (E′, A, and B are shown; E, A′, and B′ are the reciprocal). Curved arrow indicates the site of ligation. Solid boxes indicate critical wobble pairs that provide enhanced catalytic activity compared to the parental R3C ligase. Dashed boxes indicate paired regions and catalytic nucleotides that were altered to construct various cross replicators. (C) Variable portion of 12 different E enzymes. Four nucleotides at the 5′ and 3′ ends of the enzyme were chosen as the sites for genotypic variation, and 11 nucleotides within the catalytic core were chosen as the corresponding sites for phenotypic variation (boxed regions). The corresponding E′ enzymes have a complementary sequence in the paired region and the same sequence of catalytic nucleotides (alterations of the catalytic core relative to the E1 enzyme are highlighted by black circles).

FIG. 2. Self-sustained amplification of cross-replicating RNA enzymes. (A) The yield of both E (black) and E′ (gray) increased exponentially before leveling off as the supply of substrates became exhausted. (B) Amplification was sustained by performing a serial transfer experiment, allowing about 25-fold amplification before transferring 1/25th of the mixture to a new reaction vessel that contained a fresh supply of substrates. The concentrations of E and E′ were measured at the end of each incubation.

FIG. 3. Catalytic activity and exponential amplification of 12 pairs of cross-replicating RNA enzymes. (A) For each pair, the observed rate of E (black) and E′ (gray) was measured in a reaction mixture containing 5 μM E (or E′), 0.1 μM [5′-³²P]-labeled A′ (or A), 6 μM B′ (or B), 15 mM MgCl₂, and 50 mM EPPS (pH 8.5), which was incubated at 30° C. Values for k_{obs} were determined as described above. (B) For exponential amplification, the yield of newly-synthesized E and E′ relative to the starting amount of each enzyme was determined following incubation at 42° C. for 5 hours in a reaction mixture containing 0.1 μM each E and E′, 5 μM each [5′-³²P]-labeled A and A′, 5 μM each B and B′, 15 mM MgCl₂, and 50 mM EPPS (pH 8.5).

FIG. 4. Serial transfer experiment initiated by cross-replicating RNA enzymes E1-E4 and their partners E1′-E4′. (A) Amplification was sustained for 16 successive rounds of about 20-fold amplification and 20-fold dilution. The concentrations of all E (black) and E′ (gray) molecules were measured at the end of each incubation. (B) Observed genotypes among 25 E′ clones that were sequenced following the last incubation. (C) Estimated ΔΔG values for binding of each possible combination of A•B′, A•B, A′•B′, A′•B pairings relative to the corresponding matched interaction (dashes). It is difficult to calculate ΔG values in the context of the enzyme-substrate complex, but ΔΔG values only consider relative predicted binding energy for the paired region, based on values obtained from the m-fold web server at Rensselaer Polytechnic Institute (Mathews et al., 1999; Zuker, 2003). ΔΔG values that are <3.5 kcal/mol are highlighted in red. (D) Preferred pathways for mutation among B (and B′) substrates and among A′ substrates, corresponding to the most favorable ΔΔG values for mismatched pairings shown in FIG. 4C.

FIG. 5. Self-sustained amplification of a population of cross-replicating RNA enzymes, resulting in selection of the fittest replicators. (A) Beginning with 12 pairs of cross-replicating RNA enzymes (FIG. 1C), amplification was sustained for 20 successive rounds of about 20-fold amplification and 20-fold dilution. The concentrations of all E (black) and E′ (gray) molecules were measured after each incubation. (B) Graphical representation of 50 E and 50 E′ clones (dark and light columns, respectively) that were sequenced following the last incubation. The A and B (or B′ and A′) components of the various enzymes are shown on the horizontal axes, with non-recombinant enzymes indicated by shaded boxes along the diagonal. The number of clones containing each combination of components is shown on the vertical axis. (C) Exponential amplification of the starting cross-replicating enzymes E and E′ and of the most-efficient cross-replicator (A5B3 and B5′A3′) that emerged during serial transfer involving all 48 substrates. Comparative growth of E1 (circles) and A5B3 (squares) in the presence of either their cognate substrates alone (filled symbols) or all substrates that were present during serial transfer (open symbols). (D) Growth of A5B3 (black) and B5′A3′ (gray) in the presence of the eight substrates (A5, B2, B3, B4, B5′, A2′, A3′, and A4′) that comprise the three most abundant cross-replicating enzymes.

FIG. 6. Sequence and secondary structure of autocatalytic aptazymes. The complex shown is that of the enzyme E and its substrates A′ and B′. Curved arrow indicates the site of ligation, resulting in formation of E′. The reciprocal reaction, involving the enzyme E′ and substrates A and B, is not shown. Dashed boxes indicate regions that were replaced by either the theophylline or FMN aptamer to form the corresponding aptazymes. Solid boxes indicate regions of Watson-Crick pairing that were replaced to allow multiplexed exponential amplification (the AAGU sequence in A′ was replaced by AGUA; the UGAA sequence in B′ was replaced by AUGA).

FIG. 7. Ligand-dependent RNA-catalyzed ligation of RNA. In the presence of 5 mM theophylline, the aptazyme Eₜₕₑₒ catalyzed the ligation of A′ₜₕₑₒ and B′ to form E′ₜₕₑₒ (gray), and the aptazyme E′ₜₕₑₒ catalyzed the ligation of Aₜₕₑₒ and B to form Eₜₕₑₒ (black). There was no detectable activity in the absence of theophylline or in the presence of 5 mM caffeine. Reaction conditions: 5 μM Eₜₕₑₒ or E′ₜₕₑₒ, 0.1 μM [5-³²P]-labeled A′ₜₕₑₒ or Aₜₕₑₒ, 6 μM B′ or B, 25 mM MgCl₂, and 50 mM EPPS (pH 8.5) at 42° C.

FIG. 8. Ligand-dependent exponential amplification of RNA. (A) The theophylline-dependent aptazymes, Etheo (black) and E′theo (gray), amplified exponentially in the presence of 5 mM theophylline (filled circles), but not in the presence of 5 mM caffeine (open circles). The structures of theophylline and caffeine are shown. (B) Exponential growth rate of Etheo in the presence of various concentrations of theophylline. (C) The FMN-dependent aptazymes, EFMN (black) and E′FMN (gray), amplified exponentially in the presence of 1 mM FMN. The structure of FMN is shown. (D) Exponential growth rate of E_{FMN} in the presence of various concentrations of FMN. Growth rates for reactions that did not proceed beyond 10% fraction reacted were determined by a linear rather than exponential fit. (E) Time course of theophylline-dependent reaction was plotted to determine the exponential growth rate. (F) Time course of FMN-dependent reaction was plotted to determine the exponential growth rate.

FIG. 9. Sustained ligand-dependent exponential amplification of RNA. The theophylline-dependent aptazymes underwent three successive rounds of exponential amplification over 5 hours, transferring 1% of the material from a completed round to initiate the next round. Reaction conditions: 0.02 μM Eₜₕₑₒ and E′ₜₕₑₒ (first round only), 5 μM Aₜₕₑₒ, A′ₜₕₑₒ, B, and B′, 5 mM theophylline, 25 mM MgCl₂, and 50 mM EPPS (pH 8.5) at 42° C.

FIG. 10. Exponential amplification dependent on the presence of two different ligands. The theophylline aptamer was installed in enzyme E and substrate A, and the FMN aptamer was installed in enzyme E′ and substrate A′. Exponential growth occurred in the presence of both ligands (filled circles), but only linear amplification occurred in the presence of either theophylline or FMN alone (half-filled circles). Similar results were obtained when the theophylline aptamer was installed in E′ and A′ and the FMN aptamer was installed in E and A (data not shown). Reaction conditions: 0.02 μM Eₜₕₑₒ and E′_{FMN}, 5 μM Aₜₕₑₒ, A′_{FMN}, B, and B′, 2 mM theophylline and/or 1 mM FMN, 25 mM MgCl₂, and 50 mM EPPS (pH 8.5) at 42° C.

FIG. 11. Multiplexed ligand-dependent exponential amplification of RNA. The theophylline- and FMN-dependent aptazymes were made to contain distinct regions of Watson-Crick pairing. Exponential amplification of Eₜₕₑₒ (circles) and E_{FMN} (squares) occurred in the presence of both ligands (black) and in the presence of their cognate ligand alone (gray), but not in the presence of the non-cognate ligand alone (open symbols). Reaction mixtures contained 0.1 μM Eₜₕₑₒ and E′ₜₕₑₒ, 0.02 μM E_{FMN} and E′_{FMN}, and 5 μM each of the eight corresponding RNA substrates.

FIG. 12. Monitoring the course of exponential amplification by a luciferase assay, driven by the release of inorganic pyrophosphate that accompanies RNA ligation. Amplification was carried out in the presence of 5 mM theophylline, and the summed yields of Eₜₕₑₒ and E′ₜₕₑₒ were measured both by separating the ligated products in a denaturing polyacrylamide gel (filled circles) and based on the luminescent signal generated by an ATP-regenerative luciferase assay (filled squares) (Ronaghi et al., 1996). Light units were converted to absolute concentrations of inorganic pyrophosphate based on comparison to known standards. There was no light signal above background in the absence of theophylline (open squares); slightly negative values are due to imprecision in determining the conversion factor.

FIG. 13. Calibration of pyrophosphate-dependent luminescent signal in the ATP-regenerative assay based on analysis of standard concentrations of inorganic pyrophosphate. The best-fit line had a slope of 260 light units per μM pyrophosphate (r=0.999).

FIG. 14. Ligand-dependent exponential amplification of RNA in the presence of deproteinized bovine calf serum. The theophylline-dependent enzymes Eₜₕₑₒ and E′ₜₕₑₒ exhibited exponential growth rates of 0.97 and 082 h⁻¹, respectively, similar to that observed in the absence of calf serum. Reaction conditions: 0.02 μM E_{FMN} and E′_{FMN}, 5 μM of the four corresponding RNA substrates, 5 mM theophylline, 25 mM MgCl₂, 50 mM EPPS (pH 8.5), 1 U/μL Superasin and 10% phenol extracted bovine calf serum at 42° C.

### DETAILED DESCRIPTION OF THE INVENTION

### Definitions

As used herein, “self-replicating molecules” are molecules that function as both template and replicative machinery. For example, a ribozyme may be prepared that ligates two substrates (A and B) that correspond to the 5′ and 3′ portions of the ribozyme itself. The resulting enzyme-product complex must then dissociate to make available two ribozyme molecules that can enter the next cycle of replication. The 5′-terminal portion of A and the 3′-terminal portion of B, both of which are bound by the ribozyme, are complementary to each other. However, since A and B can bind to each other in an intermolecular fashion, and the corresponding portions of T can bind to each other in an intramolecular fashion, both potentially limit the rate of self-replication. In contrast, a cross-catalytic system involving two ribozymes that catalyze each other's synthesis from a total of four component substrates can replace the self-complementary relationship between A and B with cross-complementary relationships between A and B′ and between A′ and B. Thus, because ribozyme T catalyzes the ligation of A′ and B′ to form T′, and the ribozyme T′ catalyzes the ligation of A and B to form T, the ribozymes T and T′ would no longer be self-complementary at their termini.

As used herein, the term “base pair” (bp) is generally used to describe a partnership of adenine (A) with thymine (T) or uracil (U), or of cytosine (C) with guanine (G), although it should be appreciated that less-common analogs of the bases A, T, C, and G may occasionally participate in base pairings. Nucleotides that normally pair up when DNA or RNA adopts a double stranded configuration may also be referred to herein as “complementary bases”.

As used herein, the term “biosensor” refers to an analytical tool containing biologically active materials, such as enzymes or antibodies, used in conjunction with a device that will translate a biochemical interaction of those enzymes or antibodies with a target into a quantifiable signal such as light or electric pulse. Biosensors are useful in the detection of small molecules, protein targets and whole cells for diagnostic purposes. Biological systems utilized by biosensors include whole cell metabolism, ligand binding and antibody-antigen reactions. The term “biodetection” refers to the biosensor activity of detecting small molecules, protein targets, or entire cells.

As used herein, “chimeric” means a structure comprising nucleic acid from at least two different species, such as ribonucleic acid and deoxyribonucleic acid. “Chimeric” also means a structure comprising DNA or RNA which is linked or associated in a manner which does not occur in the “native” or wild type of the species.

“Complementary nucleotide sequence” or a “complementary sequence” generally refers to a sequence of nucleotides in a single-stranded molecule of DNA or RNA that is sufficiently complementary to that on another single strand to specifically hybridize to it with consequent hydrogen bonding.

As used herein, the term “isolated” refers to in vitro preparation and isolation of a synthetic product, e.g., nucleic acid, from association with other components that is associated with, e.g., components of a reaction mixture. For example, an “isolated nucleic acid molecule” includes a polynucleotide of genomic, cDNA, RNA, or synthetic origin or some combination thereof. An isolated nucleic acid molecule means a polymeric form of nucleotides of at least 2 bases in length, at least 5 bases in length, or at least 10 bases in length, either ribonucleotides or deoxyribonucleotides or a modified form of either type of nucleotide. The term includes single and double stranded forms of DNA.

As used herein, “kcat” is a rate constant corresponding to the slowest step or steps in the overall catalytic pathway. It represents the maximum number of molecules of substrate which can be converted into product per enzyme molecule per unit time. Kcat is often known as the turnover number.

As used herein, “Kₘ” refers to the Michaelis-Menten constant for an enzyme, defined as the concentration of the specific substrate at which a given enzyme yields one-half its maximum velocity in an enzyme catalyzed reaction. The values give a useful indication of the affinity of the enzyme for the involved substrate.

As used herein, a “ligase” is a nucleic acid sequence that is capable of catalyzing the covalent joining of a substrate to the same or another substrate, e.g., another nucleic acid such as a RNA sequence.

“Nucleotide” generally refers to a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a “nucleoside”. When the nucleoside contains a phosphate group bonded to the 3′ or 5′ position of the pentose, it is referred to as a nucleotide. A sequence of operatively linked nucleotides is typically referred to herein as a “nucleotide sequence”, and grammatical equivalents, and is represented herein by a formula whose left to right orientation is in the conventional direction of 5′-terminus to 3′-terminus, unless otherwise specified.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. An oligonucleotide can include a label for detection, if desired.

“Oligonucleotide” generally refers to a polymer of single- or double-stranded nucleotides. As used herein, “oligonucleotide” and its grammatical equivalents will include the full range of nucleic acids. An oligonucleotide will typically refer to a nucleic acid molecule comprised of a linear strand of naturally occurring and modified nucleotides linked together by naturally occurring and non-naturally occurring oligonucleotide linkages. An oligonucleotide may be chimeric. An oligonucleotide may comprise both RNA and DNA components. The exact size will depend on many factors, which in turn depends on the ultimate conditions of use, as is well known in the art. Oligonucleotides of the invention can be either sense or antisense oligonucleotides.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers comprising at least 7-8 nucleotides. These primers can be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. Thus, PCR-based cloning approaches rely upon conserved sequences deduced from alignments of related gene or polypeptide sequences.

As used herein, the term “prime” or “priming” means to fill the microfluidic circuit with fluid in order to prepare the circuit for subsequent steps. In some embodiments, the priming step comprises the addition of a population of ribozymes, or double-stranded DNA encoding ribozymes, or cDNA, or other “seed,” to the circuit. Subsequently, diluent/reaction mixture is added to the circuit and mixing occurs. Alternatively, the circuit may be primed with the reaction mixture prior to the addition of the DNA or RNA seed.

As used herein, the term “progeny nucleic acid molecules” describes molecules that are generated after one or more rounds of in vitro evolution seeded with a “parent” nucleic acid molecule. Progeny nucleic acid molecules may include one or more mutations not typically found in the parent nucleic acid molecules. In various alternative embodiments, a progeny nucleic acid molecule may have any number or combination of various mutations, which may be caused by mutagenic conditions employed in the methods. For example, “progeny ribozymes” are generated after one or more rounds of in vitro evolution seeded with a “parent” ribozyme. Progeny ribozymes may include one or more mutations not typically found in the parent ribozymes. In various alternative embodiments, a progeny ribozyme may have any number or combination of various mutations, which may be caused by mutagenic conditions employed in the methods.

As used herein, the term “ribozyme” or “RNA enzyme” is used to describe an RNA-containing nucleic acid that is capable of functioning as an enzyme. In the present disclosure, the term “ribozyme” includes endoribonucleases and endodeoxyribonucleases. The term “ribozyme” encompasses an RNA sequence that has ligase activity; that is, being capable of catalyzing the covalent joining of a substrate to the ribozyme or of two or more substrates. The term “ribozyme” also encompasses amide bond- and peptide bond-cleaving nucleic acid enzymes. Other terms used interchangeably with ribozyme may include “enzymatic RNA.” A “catalytic RNA population” may be a sample of homogenous catalytic RNAs, or can be a heterogeneous sample of catalytic RNAs. Catalytic or enzymatic RNA molecules of the present invention may have ligase, amide-cleaving, amide bond-cleaving, amidase, peptidase, or protease activity, or any combination thereof. These terms may be used interchangeably herein.

Ribozymes may be chosen from group I, II, III, or IV introns. Other enzymatic RNA molecules of interest herein are those formed in ribozyme motifs known in the art as “hammerhead” and “hairpin”.

As used herein, a “substrate” is defined as a molecule that may be acted upon by a nucleic acid molecule of the invention, e.g., a ribozyme. In some embodiments described herein, the substrate is an oligonucleotide. In some embodiments described herein, the substrate is a chimeric oligonucleotide. The substrate may comprise RNA, modified RNA, an RNA-DNA polymer, a modified RNA-DNA polymer, a modified DNA-RNA polymer or a modified RNA-modified DNA polymer. RNA contains nucleotides comprising a ribose sugar and adenine, guanine, uracil or cytosine as the base at the 1′ position. Modified RNA contains nucleotides comprising a ribose sugar and adenine, thymine, guanine or cytosine and optionally uracil as the base. An RNA-DNA polymer contains nucleotides containing a ribose sugar and nucleotides containing deoxyribose sugar and adenine, thymine and/or uracil, guanine or cytosine as the base attached to the 1′ carbon of the sugar. A modified RNA-DNA polymer is comprised of modified RNA, DNA and optionally RNA (as distinguished from modified RNA). Modified DNA contains nucleotides containing a deoxyribose or arabinose sugar and nucleotides containing adenine, uracil, guanine, cytosine and possibly thymine as the base. A modified DNA-RNA polymer contains modified DNA, RNA and optionally DNA. A modified RNA-modified DNA polymer contains modified RNA-modified DNA, and optionally RNA and DNA.

“Substrate specificity,” as used herein, refers to the specificity of an enzymatic nucleic acid molecule for a particular substrate, such as one comprising ribonucleotides only, deoxyribonucleotides only, or a composite of both. Substrate molecules may also contain nucleotide analogs. In various embodiments, an enzymatic nucleic acid molecule may bind to a particular region of a hybrid or non-hybrid substrate.

“Ligand specificity,” as used herein, refers to the binding specificity of a portion of an enzymatic nucleic acid molecule of the invention for a particular ligand, which may be a nucleic acid molecule, protein or other biological molecule, or any nonbiological molecule, e.g., a synthetic molecule.

### Evolution of RNA Enzymes of the Invention

One of the most enduring questions is how life could have begun on Earth. Molecules that can make copies of themselves are thought to be crucial to understanding this process as they provide the basis for heritability, a critical characteristic of living systems. As described below, a significant step toward answering that question has been taken, as RNA enzymes that can replicate themselves without the help of any proteins or other cellular components, which process proceeds indefinitely, have been prepared.

In the modern world, DNA carries the genetic sequence for advanced organisms, while RNA is dependent on DNA for performing its roles such as building proteins. But one prominent theory about the origins of life, called the RNA World model, postulates that because RNA can function as both a gene and an enzyme, RNA might have come before DNA and protein and acted as the ancestral molecule of life. However, the process of copying a genetic molecule, which is considered a basic qualification for life, appears to be exceedingly complex, involving many proteins and other cellular components. For years, researchers have wondered whether there might be some simpler way to copy RNA, brought about by the RNA itself. Using a method of forced adaptation, i.e., in vitro evolution, a RNA enzyme that could replicate was improved so that it could drive efficient, perpetual self-replication.

A large population of variants of the RNA enzyme was synthesized and test-tube evolution employed to obtain variants that were most adept at joining together pieces of RNA. Ultimately, this process led to an evolved version of the original enzyme that is a very efficient replicator. The improved enzyme was able to undergo perpetual replication.

The replicating system involves two enzymes, each composed of two substrates and each functioning as a catalyst that assembles the other. The replication process is cyclic, in that the first enzyme binds the two substrates that include the second enzyme and joins them to make a new copy of the second enzyme; while the second enzyme similarly binds and joins the two substrates that include the first enzyme. In this way the two enzymes assemble each other, what is termed cross-replication. To make the process proceed indefinitely requires only a small starting amount of the two enzymes and a steady supply of the substrates.

A variety of enzyme pairs with similar capabilities was also generated. Twelve different cross-replicating pairs were mixed, together with all of the constituent substrates, and allowed to compete in a molecular test of survival of the fittest. Most of the time the replicating enzymes bred true, but on occasion an enzyme would bind one of the substrates from one of the other replicating enzymes. When such “mutations” occurred, the resulting recombinant enzymes also were capable of sustained replication, with the most fit replicators growing in number to dominate the mixture. The system can sustain molecular information, a form of heritability, and give rise to variations of itself in a way akin to Darwinian evolution.

### Evolving RNA Enzymes

The principles of Darwinian evolution are fundamental to understanding enzymatic function, and have been applied to the development of novel enzymes in the test tube. Laboratory evolution is greatly accelerated compared to natural evolution, but typically requires substantial manipulation by the experimenter. A system that relies on computer control and microfluidic chip technology was developed to automate the directed evolution of functional molecules, subject to precisely defined parameters (see PCT/US06/039733 and PCT/US06/039594, the disclosures of which are incorporated by reference herein).

A population of billions of RNA enzymes with RNA-joining activity was challenged to react in the presence of progressively lower concentrations of substrate. The reacted enzymes were amplified to produce progeny, which were challenged similarly. Whenever the population size reached a predetermined threshold, chip-based operations were executed to isolate a fraction of the population and mix it with fresh reagents. These steps were repeated automatically for 500 iterations of 10-fold exponential growth followed by 10-fold dilution. Evolution was observed in real time as the population adapted to the imposed selection constraints and achieved progressively faster growth rates over time.

The microfluidic system relies on polymerase proteins to bring about the selective amplification of RNA. More recently, RNA enzymes were developed that have the ability to catalyze their own replication in the absence of proteins or any other biological materials (Kim and Joyce, 2004). The “R3C” RNA enzyme is an RNA ligase that binds two oligonucleotide substrates through Watson-Crick pairing and catalyzes nucleophilic attack of the 3′-hydroxyl of one substrate on the 5′-triphosphate of the other, forming a 3′,5′-phosphodiester and releasing inorganic pyrophosphate. The R3C ligase was configured to self-replicate by joining two RNA molecules to produce another copy of itself (Paul and Joyce, 2002). This process was inefficient because the substrates formed a non-productive complex that limited the extent of exponential growth, with a doubling time of about 17 hours and no more than two successive doublings.

An RNA enzyme that catalyzes the RNA-templated joining of RNA was converted to a format whereby two enzymes catalyze each other's synthesis from a total of four component substrates (Kim and Joyce, 2004). As described herein below, these cross-replicating RNA enzymes were optimized so that they can undergo self-sustained exponential amplification at a constant temperature. Amplification occurs with a doubling time of about one hour, and can be continued indefinitely. Populations of various cross-replicating enzymes were constructed and allowed to compete for a common pool of substrates. During a serial transfer experiment in which the population underwent overall amplification of >10²⁵-fold, recombinant replicators arose and grew to dominate the population. RNA enzymes that undergo self-sustained replication can serve as an experimental model of a genetic system. Many such model systems could be constructed, allowing different selective outcomes to be related to the underlying properties of the genetic system.

### Serial Dilution

Serial dilution is among the most fundamental and widely practiced laboratory techniques, with applications ranging from generating sets of standards, to performing in vitro evolution, to culturing cells. Performing serial dilutions by manual pipetting is a mundane and time-consuming task that has limited the execution of highly longitudinal experiments in molecular evolution. Microfluidic technology presents a practical solution to this problem by automating the fluid handling associated with serial dilution.

The core strengths of microfluidic technology are integration, high throughput, and low-volume handling. Microfluidic analogs outperform conventional instrumentation with regard to speed, throughput, and reagent consumption by an order of magnitude or more, and allow integration of sample preparation and analysis in a single device. Precise manipulation of fluids in these devices may be achieved by electrokinetic control, microfabricated membrane valves, or various other approaches to microfluidic transport and control. The combination of highly ordered flow and precise manipulation allows one to carry out diverse synthetic and analytical methods with remarkable control.

A microfluidic serial dilution circuit that implements these advantageous mixing and scaling characteristics and incorporates sample metering elements has been designed, fabricated, and characterized (see PCT/US06/039733). Use of such a system can be employed on the nanoliter scale and does not geometrically constrain the number of possible serial dilutions. Precise metering of the sample carryover fraction and rapid, reproducible mixing of the diluent with the carryover are achieved in the same structure. The methods employing the circuit may be computer controlled, and the preparation of successive serial dilutions may be fully automated. Fluidic operations, such as diluent flushing, mixing, and priming can be accurately and precisely performed without manual intervention, and performed simultaneously in many parallel circuits. Because the methods employ microfluidic pumping, serially diluted sample aliquots can easily be routed from the dilution circuit to other microfluidic components, such as a separation channel or microreactor.

### Microfluidic-Based Continuous In Vitro Evolution

Serial dilution is employed in directed evolution experiments in which a population of RNA molecules is made to undergo repeated rounds of selective amplification. In order to evolve molecules with desired properties, the population of RNAs is propagated through many logs of selective growth. This may be accomplished by serially diluting an aliquot of the reaction mixture into fresh reaction medium at regular intervals.

The methods described herein combine biochemical systems for the continuous in vitro evolution of RNA enzymes using microfluidic technology. They allow Darwinian evolution to be carried out much more rapidly and precisely, and using smaller volumes of reagents, than pipettes and PAGE analysis, with complete control over variables such as population size, mutation frequency, and selection pressure.

Continuous in vitro evolution of an RNA ligase is accomplished by challenging a population of RNA molecules in the circuit to perform a desired reaction. In an embodiment, the RNA molecules ligate to their own 5′ end an oligonucleotide substrate that contains the sequence of an RNA polymerase promoter element. Molecules that successfully ligate are reverse transcribed to cDNAs that contain a functional promoter, which in turn are transcribed to generate “progeny” ribozymes.

As Darwinian evolution proceeds, mutations are acquired, the catalytic efficiency of ribozymes in the population improves, and the time for reaching a pre-determined nucleic acid threshold concentration becomes shorter. The catalytic efficiency (kcat/Kₘ) of the ribozymes increases, and the doubling time for selective amplification decreases. Small aliquots of the growing population are serially diluted in the circuit into a new reaction mixture that contains a fresh supply of the substrate and polymerase enzymes. Reproduction is selective for RNA molecules with ligase activity, and mutations accumulate through error-prone enzymatic replication.

It is contemplated that any RNA molecule capable of ligating a substrate to itself can be employed in the methods described herein. In certain embodiments, the enzymatic RNA molecule is derived from a group I, II, III, or IV intron. In another variation, an enzymatic RNA molecule contemplated herein comprises the portions of a group I, II, III or IV intron having catalytic activity.

In one embodiment of continuous in vitro evolution, evolved variants are from group I ligase ribozymes. This ribozyme catalyzes the template-directed joining of an oligonucleotide 3′-hydroxyl and an oligonucleotide 5′-triphosphate, forming a 3′,5′-phosphodiester and releasing inorganic pyrophosphate.

The nucleic acid material that is subjected to evolution that is used to start or “seed” the reaction can include, but is not limited to, an isolated population of ribozymes; the substrate(s) of a ribozyme; a dsDNA copy of the ribozyme (i.e., a PCR product); a single-stranded cDNA (i.e., the complement of the ribozyme); the products of a previous burst of continuous evolution; or any combination thereof.

The nucleic acid material that is subjected to evolution may be introduced into the microfluidic device at starting concentrations ranging from about 0.1 nM-10 μM, e.g., from about 1 nM to 1 μM or from about 10 nM-100 nM.

The nucleotide substrate(s) that is/are acted upon by a ribozyme can be introduced into the microfluidic device at starting concentrations ranging from about 0.1 nM-1 mM, e.g., about 1 nM-100 μM or about 10 nM-10 μM.

Various embodiments of the disclosed invention contemplate that an enzymatic RNA molecule that includes one or more mutations not typically found in wild-type enzymatic RNA molecules or ribozymes. In various alternative embodiments, an enzymatic RNA molecule of the present invention may have any number or combination of the various disclosed mutations. For example, a catalytic RNA molecule of the present invention may have 1-5 mutations, 1-10 mutations, 1-15 mutations, 1-20 mutations, 1-25 mutations, 1-30 mutations, or even more. It should be understood that mutations need not occur in 5-mutation increments. The invention contemplates that any number of mutations may be incorporated into catalytic RNA molecules of the present invention, as long as those mutations do not interfere with the molecules' ability to ligate substrates.

As a person of skill in the art will appreciate, nearly every parameter of the continuous evolution methods described herein can be modified by the researcher in order to direct the evolution and generate ribozymes having specific activities. Indeed, vast ribozyme diversity and specificity can be obtained by any number of alterations or selective pressures applied to the system. Depending on the purpose and desired outcome of the experiment, the concentrations of ribozyme, reaction mixture ingredients including substrate, enzymes, and buffer components, can be varied within effective ranges that are known by those of skill in the art.

The methods described herein can be conducted at higher or lower temperatures. The test RNA seed may be used to initially prime the system, or may be added in the diluent flush. Likewise, the reaction buffer containing the substrate may be used to initially prime the system or alternatively may be added in the diluent flush.

The dilution carried out can be varied or kept constant, and is essentially unlimited. The fluid in the circuit can be diluted by the diluent reaction mixture about 1:1, about 1:10, about 1:100, about 1:1000, about 1:10,000, and so on. In one embodiment, continuous in vitro evolution is conducted using a series of dilutions of about 1:10 to take advantage of the high rate of reaction that occurs under those conditions.

Depending on the application, suitable circuit mixing times range from about 0.1 seconds-10 minutes, e.g., about 1 second-5 minutes or about 10 seconds-1 minute.

In practice, valve actuation times can be in the range of about 0.1 millisecond-1 second, e.g., about 1 millisecond-300 milliseconds or about 10 milliseconds-100 milliseconds.

The circuit loop described herein can be scaled up or down in size, having a diameter ranging from about 0.01 cm-100 cm, e.g., about 0.1 cm-10 cm or about 0.5 cm-5 cm. Fluid channels, manifold channels, fluid reservoirs and membrane valve dimensions can be adjusted accordingly, in order to obtain effective results within these loop diameter ranges.

In practice, the circuit loop described herein could have a volume of about 1 nL-1 mL, e.g., about 10 nL-100 μL, 100 nL-10 μL or 200 nL-1 μL.

### Biosensor Applications

The methods described herein provide practical applications of microfluidic-based selective amplification, pertaining to the quantitative detection of small molecule and protein targets, such as for use in diagnostics. Amplification of target proteins or small molecules by methods including PCR, ELISA (Engvall and Perlman, 1971), and immuno-PCR (Sano et al., 1992) suffer from the fact that once exponential amplification has been initiated, it is no longer dependent on the presence of the analyte. This is beneficial for sensitivity, but not for specificity. The methods described herein allow the experimenter not only to sense the ligand dynamically during the course of amplification, but also to control and automate the system and reduce the levels of reagents consumed.

Using the methods described herein, ligase aptazymes can be optimized by being subjected to continuous evolution in a ligand-dependent manner. The concentration of the cognate ligand can be adjusted to control the evolutionary fitness of the continuously evolving ribozymes. These ribozymes can be isolated and analyzed and can subsequently be used to detect small molecule and protein targets that are relevant to analytical biochemistry, environmental monitoring, and other biosensor applications.

It is contemplated that the methods described herein may be further employed in biosensor applications including but not limited to: glucose monitoring in diabetes patients; measuring other constituents of blood such as S-adenosylhomocysteine; detecting health related targets, such as amyloid peptide; environmental applications such as the detection of pesticides and river water contaminants; remote sensing of airborne bacteria for example in counter-bioterrorist activities; detection of pathogens; determining levels of toxic substances before and after bioremediation; detection of organophospate, lactic acid, cholesterol, amino acids and nucleotides; detection of antibodies, phospholipases, hormones and growth factors.

### Ligand Dependent Amplification

The PCR revolutionized molecular biology and clinical diagnostics because it provided a general yet highly sequence-specific method for exponential amplification of a target nucleic acid. Although it is not possible to amplify a target small molecule or protein, methods have been devised to amplify a signal that is indicative of the presence of such compounds. The ELISA test, for example, links immunodetection of a target molecule to the multiple-turnover activity of an attached enzyme (e.g., horseradish peroxidase), resulting in linear amplification of an optically detectable signal (Engvall and Derlman, 1970). Other methods link immunodetection to exponential amplification by employing an antibody-DNA conjugate, the DNA portion of which is amplified by either the PCR or ligase chain reaction (Sano et al., 1992; Fredriksson et al., 2002). All of these amplification technologies, including the PCR, suffer from the fact that once exponential amplification has been initiated, it is no longer dependent on the presence of the analyte. This is beneficial for sensitivity, but not for specificity. In some cases it would be preferable to sense the ligand dynamically throughout the course of amplification. Furthermore, it would be beneficial to have an amplification method that does not depend on a protein (e.g., a DNA polymerase) and does not require temperature cycling.

Aptazymes are RNA (or DNA) enzymes whose activity is dependent on the recognition of a target ligand. The catalytic domain of the enzyme is connected to a ligand binding domain such that activity of the enzyme is greatly enhanced upon binding of the cognate ligand (Tang and Breaker, 2005). A ligand binding domain composed of RNA (or DNA) is referred to as an “aptamer”. Some aptamers occur in nature as regulatory elements within messenger RNA (“riboswitches”) (Tucker and Breaker, 1997), but most have been developed in the laboratory using methods of in vitro evolution (Fitzwater and Polisky, 1996; Ciesioeka, 1996). Aptamers may be obtained by constructing a library of random-sequence RNAs and carrying out repeated rounds of selective amplification to discover particular RNAs that bind tightly and specifically to the target ligand. Aptamers typically contain 20-50 nucleotides and bind their cognate ligand with a K_{d} of 10⁻⁵-10⁻¹⁰ M. Aptamers have been developed to bind a diverse array of targets ranging from small molecules to proteins, and even whole cells (Morris et al., 1998). The generation of aptamers for a wide variety of ligands has had many applications in biotherapeutics, medical diagnostics, and biosensing (Rimmell, 2003; Brody and Gold, 2000; Ng et al., 2000). Aptazymes also have been used in diagnostics and biosensing, where the activity of the enzyme provides a signal that is indicative of the presence of the ligand (Seetharamin et al., 2001; Hesselberth et al., 2003; Hartig et al., 2002; Vaish et al, 2002). For example, the class I ligase ribozyme has been made to operate as an aptazyme that is dependent on a target viral nucleic acid for its activity (Vaish et al., 2003; Kossen et al., 2004). The ribozyme ligates two oligonucleotide substrates in the presence, but not the absence, of the target, and undergoes multiple turnovers to provide linear signal amplification that depends on ongoing target recognition. Other ligase ribozymes have been made to operate as aptazymes that are dependent on either a small molecule or protein ligand, albeit without catalytic turnover (Robertson and Ellington, 2001; Robertson et al., 2004).

One well-studied class of RNA enzymes are the RNA ligases, which catalyze the RNA-templated joining of RNA molecules. Some RNA ligases have been made to operate as aptazymes, and some of these have been made to undergo ligand-dependent catalytic turnover to provide linear signal amplification with ongoing target recognition (Hartig et al., 2002; Vaish et al., 2002). One of the known RNA ligases is the “R3C” RNA enzyme, which was obtained using in vitro evolution (Rogers and Joyce, 2001). This enzyme has been reconfigured so that it can self-replicate by joining two RNA molecules that result in formation of another copy of itself (Paul and Joyce, 2002). It also has been converted to a cross-catalytic format, whereby two RNA enzymes catalyze each other's synthesis from a total of four RNA substrates. The cross-replication process is analogous to the ligase chain reaction, except that in cross-replication the nucleic acid being amplified is itself the ligase, and strand separation occurs spontaneously without requiring temperature cycling.

The activity of the cross-replicating RNA enzymes has been greatly improved so that they can undergo efficient exponential amplification, generating about a billion copies in 30 hours at a constant temperature of 42° C. (see Example 1). Exponential amplification can be continued indefinitely; so long as a supply of the four substrates is maintained. The reaction does not require any proteins or other biological materials. Millimolar concentrations of Mg²⁺ (e.g., 5-25 mM) support the activity of the RNA enzymes, and the reaction mixture is buffered to maintain an appropriate pH (e.g., pH 7.5-8.5).

Autocatalytic aptazymes undergo exponential amplification dependent on the presence of a target ligand. As with simple aptazymes, an aptamer domain is connected to the catalytic domain of a cross-replicating enzyme. Because new copies of the enzymes are generated from the four RNA substrates, one or more of these substrates contain the aptamer domain. A small number of enzymes that are present at the outset are amplified to generate a vast number of copies, but exponential amplification only occurs if the ligand is present. This gives rise to a large signal that is readily distinguished from the background when no ligand is present. The signal may be the newly-formed enzymes themselves, or some measurable property that reflects their formation, such as a fluorescent or luminescent signal associated with the ligated products. For example, the enzyme ATP sulfurylase quantitatively converts pyrophosphate to ATP, which in turn drives a luciferase-mediated conversion of luciferin to oxyluciferin to generate visible light. Alternatively, the aptamer or ligand may be labeled, e.g., with a fluorescent label and the amount of that label, e.g., incorporated into or bound to the aptazyme, detected. Thus, using autocatalytic aptazymes, a fluorescent or luminescent reporter of exponential amplification may be based on the release of inorganic pyrophosphate, which occurs with each ligation event.

### Exemplary Embodiments

The R3C ligase was converted to an aptazyme by replacing the distal portion of the central stem-loop by an aptamer domain that specifically binds theophylline. Theophylline has a molecular weight of 180 g/mol and is commonly used as a bronchodilator for the treatment of asthma and chronic obstructive pulmonary disease. The theophylline aptamer binds theophylline with high affinity, but it binds poorly to caffeine which differs from theophylline by only a methyl group. The activity of the R3C aptazyme was found to be strongly dependent on the presence of theophylline, but was not activated by caffeine. The level of activity in the presence of theophylline, and the ratio of activity in the presence compared to the absence of theophylline, could be adjusted by varying the stability of the stem that connects the aptamer domain to the catalytic domain of the aptazyme.

The aptamer domain was installed into one of the two substrates that gives rise to each of the two cross-replicating enzymes. All four substrates were provided at 5 μM concentration and 0.02 μM of each enzyme was used as a seed for exponential amplification. The reaction mixture also contained 25 mM MgCl₂ and 25 mM EPPS buffer at pH 8.5. Either 5 mM theophylline or 5 mM caffeine was added to the mixture, which was maintained at a constant temperature of 42° C. Brisk exponential amplification occurred in the mixture containing theophylline, but there was no detectable amplification in the mixture containing caffeine. Exponential amplification resulted in the formation of new copies of both enzymes, ultimately limited by the supply of substrates. A plot of enzyme concentration versus time exhibited a classic sigmoidal shape, indicative of exponential growth subject to a fixed supply of materials. These data were fit to the equation:

[E]ₜ=a/(I+be^{-ct}),

where [E]₁ is the concentration of enzyme at time t, a is the maximum extent of growth, b is the degree of sigmoidicity, and c is the exponential growth rate.

The exponential growth rate was determined to have a value of 0.78 hour⁻¹ in the presence of 5 mM theophylline, which corresponds to a doubling time of 0.89 hours. The maximum extent of growth was 3.3 μM due to depletion of the substrates required for exponential amplification. If a portion of the reaction mixture was transferred to a new mixture containing a fresh supply of substrates (analogous to reseeding the PCR), exponential growth could be continued indefinitely.

The exponential growth rate for cross-replicating aptazymes is dependent on the concentration of the corresponding ligand. This allows one to construct standardized curves that can be used to determine the concentration of ligand in an unknown sample. These procedures are analogous to quantitative PCR (qPCR), but can be generalized to any ligand that can be recognized by an aptamer, including small molecules and proteins.

The theophylline-dependent aptazyme was exposed to theophylline levels ranging from 0.2 to 5.0 mM and the rate of exponential growth was determined. The rate as a function of theophylline concentration provided a saturation curve that can be used to determine the concentration of theophylline in a sample. The saturation curve revealed that theophylline binds to the aptazyme with a K_{d} of 0.51 mM, and that the exponential growth rate at saturation is 0.66 hour⁻¹. Thus the aptazyme can be used to measure theophylline concentrations in the range of approximately 0.05-5 mM. In order to measure theophylline concentrations in a different concentration range one would need to employ an aptamer with a different affinity for the ligand or to employ alternative reaction conditions that shift the K_{d} of the aptamer. For example, when the theophylline-dependent aptazyme was incubated at 37° C. rather than 42° C., the K_{d} for theophylline was reduced to 0.012 mM and the exponential growth rate at saturation was 0.24 hour⁻¹.

A second autocatalytic aptazyme was constructed based on an aptamer that specifically binds flavin mononucleotide (FMN). This compound has a molecular weight of 456 g/mol and is an essential metabolite derived from vitamin B₂. Like the theophylline aptazyme, the FMN aptazyme underwent exponential amplification in the presence, but not the absence, of the ligand. The rate of exponential growth was measured in the presence of FMN concentrations ranging from 0.05 to 1.0 mM and a saturation curve was determined. It revealed that FMN binds to the aptazyme with a K_{d} of 0.068 mM, and that the exponential growth rate at saturation is 0.58 hour⁻¹. Thus the aptazyme can be used to measure FMN concentrations in the range of approximately 0.007-0.7 mM.

It is necessary that only one member of the pair be an aptazyme, the other can be a standard cross-replicating enzyme that is “always on”. Conversely, each member of the pair can be an aptazyme for a different ligand so that both ligands must be present for exponential amplification to occur. The two ligands can be different compounds or different epitopes of the same compound.

A pair of autocatalytic aptazymes was constructed in which one member of the pair contained the theophylline aptamer and the other contained the FMN aptamer. A low level of linear amplification was observed in the presence of either 2 mM theophylline or 1 mM FMN, but both ligands were required for exponential growth. A dual saturation profile could be determined by systematically varying the concentrations of the two ligands. Alternatively, a dual saturation profile could be calculated based on the saturation behavior of each of the two aptazymes that form the cross-replicating pair.

The invention will be further described by the following nonlimiting examples.

### Example 1

### Materials and Methods

Materials. Oligonucleotides were either purchased from Integrated DNA Technologies (San Diego, Calif.) or synthesized on an Expedite automated DNA/RNA synthesizer (Applied Biosystems, Foster City, Calif.) using nucleoside phosphoramidites purchased from Glen Research (Sterling, Va.). All oligonucleotides were purified by denaturing polyacrylamide gel electrophoresis (PAGE) and desalted using a C18 SEP-Pak cartridge (Waters, Milford, Mass.). Histidine-tagged T7 RNA polymerase was purified from E. coli strain BL21 containing plasmid pBH161 (kindly provided by William McAllister, State University of New York, Brooklyn). Thermus aquaticus DNA polymerase was cloned from total genomic DNA and purified as described in Pluthero et al. (1993). M1 RNA, the catalytic subunit of RNAse P, was obtained from E. coli genomic DNA (Sigma-Aldrich, St. Louis, Mo.) by PCR amplification using primers 5″-GGACTAATACGACTCACTATAGAAGCTGACCAGACAGTCG-3′ (SEQ ID NO:1) and 5″-AGGTGAAACTGACCGATAAGC-3 (SEQ ID NO:2) (T7 RNA polymerase promoter sequence underlined), followed by in vitro transcription. The PCR products were cloned into E. coli and their sequence was verified. Calf intestine phosphatase, E. coli poly(A) polymerase, and T4 polynucleotide kinase were purchased from New England Biolabs (Ipswich, Mass.), Superscript II RNase H-reverse transcriptase was from Invitrogen (Carlsbad, Calif.), and calf thymus terminal transferase was from Roche Applied Science (Indianapolis, Ind.). Nucleoside and deoxynucleoside 5′-triphosphates were purchased from Sigma-Aldrich and [γ-³²P]ATP (7 μCi/pmol) was from Perkin Elmer (Waltham, Mass.).

Preparation of RNA enzymes and substrates. All RNA enzymes and substrates were prepared by in vitro transcription. The transcription mixture contained 0.4 μM DNA template, 0.8 μM synthetic oligodeoxynucleotide having the sequence 5′-GGACTAATACGACTCACTATA-3′ (SEQ ID NO:3) (promoter sequence underlined), 2 mM each of the four NTPs, 25 U/μL T7 RNA polymerase, 15 mM MgCl₂, 2 mM spermidine, 5 mM dithiothreitol, and 50 mM Tris-HCl (pH 7.5). The mixture was incubated at 37° C. for 2 hours, then quenched by adding an equal volume of gel loading buffer containing 15 mM Na₂EDTA and 18 M urea. The transcription products were purified by PAGE, eluted from the gel, and desalted.

The A substrates could not be obtained reliably by in vitro transcription due to heterogeneity at the 3′ end of the transcripts. Instead, extended length RNAs were prepared that contained additional nucleotides, having the sequence 5′-GAGACCGCAACUUG-3′ (SEQ ID NO:4), located downstream from the A substrate sequence. The added nucleotides were removed using E. coli M1 RNA to generate a precise 3′ terminus. The cleavage reaction employed 20 μM RNA transcript, 20 μM external guide sequence RNA having the sequence 5′-GGUAAGUUGCGGUCUCACCA-3′ (SEQ ID NO:5), 5 μM M1 RNA, 100 mM MgCl₂, 100 mM NH₄Cl, and 50 mM Tris-HCl (pH 7.5). Note that the guide RNA is complementary to the extended portion of the transcript, with a 5′-terminal GG and 3′-terminal ACCA also present in the guide RNA (Forster et al., 1998). The reaction mixture was incubated at 30° C. for 8 hours, quenched, and the cleaved products were purified by PAGE, as described above. During the in vitro evolution procedure, the A′ substrates were prepared directly by in vitro transcription, but in all other instances these substrates were prepared using the M1 RNA cleavage procedure. For the A′ substrates, the added 3′-terminal nucleotides had the sequence 5′-GAGACCGCAUGAAU-3′ (SEQ ID NO:6) and the external guide sequence RNA had the sequence 5′-GGAUUCAUGCGGUCUCACCA-3′ (SEQ ID NO:7).

In vitro evolution. DNA templates used to transcribe the starting pools of B-E′ and B′-E molecules were generated by a 10-cycle PCR employing two overlapping synthetic oligodeoxynucleotides, as listed below (promoter sequence underlined; nucleotides randomized at 12% degeneracy in italics). The resulting PCR products, each consisting of about 10¹⁴ molecules, were transcribed as described above, except that it was unnecessary to provide a synthetic oligodeoxynucleotide containing the second strand of the promoter.

For B-E′
(SEQ ID NO: 8)
5′-GGACTAATACGACTCACTATAGAGACCGCAACTTAG-3′
and
(SEQ ID NO: 9)
5′-ACAGATCAGTATTCATGCGGTCTCTAAATTCAACCCATTCAAA
CTGTTCTAAGTTACCTTAGAACAATCGAGCACAACTTACTAAGTTG
CGGTCTC-3′;
For B′-E
(SEQ ID NO: 10)
5′-GGACTAATACGACTCACTATAGAGACCGCATGAATAG-3′
and
(SEQ ID NO: 11)
5′-CTTCTGGATGGTCAAGTTGCGGTCTCTTTATTCAACCC
ATTCAAACTGTTACTTACGTAACAATCGAGCACATGAACAC
TATTCATGCGGTCTC-3′.

DNA templates used to transcribe the starting pools of A and A′ molecules were prepared directly as synthetic oligodeoxynucleotides (promoter sequence underlined; nucleotides randomized at 12% degeneracy in italics). The second strand of the promoter was supplied as a synthetic oligodeoxynucleotide. The transcribed A molecules were cleaved by M1 RNA.

For A
(SEQ ID NO: 12)
5′-CAAGTTGCGGTCTCTTTATTCAACCCATTCAAACTGTTACTT
ACGTAACAATCGAGCACATGAACTCGTGTTAGCCTATAGTGA
GTCGTATTAGTCC-3′;
For A′
(SEQ ID NO: 13)
5′-TAATTCAACCCATTCAAACTGTTCTAAGTTACCTTAGAACAATC
GAGCACAACTTCAGCATAGGATTCTATAGTGAGTCGTATTAG
TCC-3′.

During each round of in vitro evolution, RNA-catalyzed RNA ligation was carried out in a reaction mixture containing 1 μM B-E′ (or B′-E), 5 μM A (or A′), 25 mM MgCl₂, and 50 mM EPPS (pH 8.5), which was incubated at 30° C. for various times. The ligated RNAs were gel purified, then reverse transcribed in a reaction mixture containing about 0.4 μM RNA, 1 μM cDNA primer, 0.5 mM each of the four dNTPs, 3 mM MgCl₂, 75 mM KCl, 10 mM dithiothreitol, and 50 mM Tris-HCl (pH 8.3), which was incubated at 37° C. for 1 hour. The resulting cDNAs were PCR amplified employing the same cDNA primer and a second primer, as listed below (promoter sequence underlined).

For A-B-E′
(SEQ ID NO: 14)
5′-GACAGATCAGTATTCATGC-3′
and
(SEQ ID NO: 15)
5′-GGACTAATACGACTCACTATAGGCTAACACGAGTTCA-3′;
For A′-B′-E
(SEQ ID NO: 16)
5′-CTTCTGGATGGTCAAGTTGC-3′
and
(SEQ ID NO: 17)
5′-GGACTAATACGACTCACTATAGAATCCTATGCTGAAGT-3′.

The PCR products were used to initiate nested PCR amplifications to generate templates for the transcription of progeny RNAs. For the B-E′ molecules, the products of this second PCR were transcribed directly. For the A molecules, it was necessary to perform three successive PCRs, rather than progressing directly from A-B-E′ to A, due to mispriming caused by sequence similarity near the 3′ ends of A and E′. The second PCR eliminated the 3′-terminal region of E′, allowing subsequent amplification of A. The products of the second PCR were incubated in the presence of 0.2 N NaOH for 20 minutes at 92° C. to bring about hydrolysis at the single ribonucleotide position, followed by neutralization with 0.2 N HCl. The shorter cleaved products were purified by PAGE and used as input for the third PCR. The products of the third PCR were transcribed to generate RNA, which was gel purified and cleaved by M1 RNA, as described above. The primers used for the various nested PCRs derived from A-B-E′ are listed below (T7 promoter underlined; ribonucleotide in bold).

For B-E′ (second PCR)
(SEQ ID NO: 18)
5′-GACAGATCAGTATTCATGC-3′
and
(SEQ ID NO: 19)
5′-GGACTAATACGACTCACTATAGAGACCGCAACTTAG-3′;
For A (second PCR)
(SEQ ID NO: 20)
5′-GACAGATCAGTATTCATGC(rG)-3′
and
(SEQ ID NO: 21)
5′-GGACTAATACGACTCACTATAGGCTAACACGAGTTCA-3′;
For A (third PCR)
(SEQ ID NO: 44)
5′-CTAAGTTGCGGTCTC-3′
and
(SEQ ID NO: 45)
5′-GGACTAATACGACTCACTATAGGCTAACACGAGTTCA-3′.

For the B′-E molecules, the products of the second PCR were transcribed directly. For the A′ molecules, the products of the second PCR were subjected to alkaline hydrolysis as described above, then the cleaved products were purified by PAGE and used as input for a third PCR. The products of the third PCR also were subjected to alkaline hydrolysis, the cleaved products were purified by PAGE, then used to transcribe the desired A′ molecules. The primers used for the various nested PCRs derived from A′-B′-E are listed below (T7 promoter underlined; ribonucleotide in bold).

For B′-E (second PCR)
(SEQ ID NO: 22)
5′-CTTCTGGATGGTCAAGTTGC-3′
and
(SEQ ID NO: 23)
5′-GGACTAATACGACTCACTATAGAGACCGCATGAATAG-3′;
For A′ (second PCR)
(SEQ ID NO: 24)
5′-CTTCTGGATGGTCAAGTTGC(rG)-3′
(SEQ ID NO: 25)
5′-GGACTAATACGACTCACTATAGAATCCTATGCTGAAGT-3′;
For A′ (third PCR)
(SEQ ID NO: 26)
5′-CTATTCATGCGGTCT(rC)-3′
and
(SEQ ID NO: 27)
5′-GGACTAATACGACTCACTATAGGAAAGAGAAAGAAGT-3′.

Six successive rounds of in vitro evolution were carried out as described above, with progressively shorter times for the RNA-catalyzed reaction:

| | | |
|---|---|---|
| Round | A-B-E′ | A′-B′-E |

| | | | | |
|---|---|---|---|---|
| 1 | 2 | h | 2 | h |
| 2 | 1 | min | 5 | min |
| 3 | 15 | s | 30 | s |
| 4 | 15 | s | 15 | s |
| 5 | 0.1 | s | 0.1 | s |
| 6 | 0.01 | s | 0.01 | s |

The last two rounds were conducted using a KinTek (Austin, Tex.) model RQF-3 quench-flow apparatus to achieve very short reaction times. Hypermutagenic PCR (Vartanian et al., 1996) was performed following round 3 to increase diversity among the population of B-E, B′-E, and A molecules. Standard mutagenic PCR (Cadwell et al., 1992) was performed following round 3 for the A′ molecules.

Following round 6, the ligated molecules were gel purified, reverse transcribed, PCR amplified, and cloned into E. coli using the Invitrogen TOPO TA Cloning Kit. The bacteria were grown on LB agar plates containing 50 μg/mL carbenicillin. Samples were taken from individual colonies and evaluated by PCR to confirm they contained plasmid DNA with an insert of the appropriate length. Validated colonies were picked from the plate and cultured overnight in 2 mL LB medium containing 50 μg/mL carbenicillin. The plasmid DNA was isolated from the cells using a QIAprep Spin Miniprep Kit (Qiagen, Valencia, Calif.), then sequenced by Genewiz Inc. (La Jolla, Calif.).

Conversion of selected enzymes to corresponding substrates. A modified version of the nested PCR amplification procedure described above can be used to produce A and B molecules from corresponding E molecules, and to produce A′ and B′ molecules from corresponding E′ molecules. In this case, B and B′ are produced as separate molecules, rather than joined to E′ and E, respectively. This requires installing a primer binding site at the 3′ end of B and B′, which also encodes a recognition sequence for the “10-23” RNA-cleaving DNA enzyme (Santoro et al., 1997). Cleavage by the DNA enzyme is used to generate transcription products with a precise 3′ terminus (Pyle et al., 2000). A and A′ are produced as above, except that they are derived from PCR-amplified E and E′, rather than A-B-E′ and A′-B′-E, respectively. In addition, the primer binding site at the 5′ end of A and A′ is shifted upstream so as not to encroach on the genotype region of these molecules.

The ligated products E and E′ are purified by PAGE, reverse transcribed, and PCR amplified, as above. A second PCR is carried out to generate templates that are used to transcribe precursor substrates that contain additional nucleotides at their 3′ terminus. The added nucleotides are removed from A and A′ using M1 RNA, as described above. The added nucleotides are removed from B and B′ using a DNA enzyme. The downstream sequences for the various substrates and corresponding external guide sequence RNA or corresponding DNA enzyme are listed below (dot indicates the site for DNA-catalyzed RNA cleavage; substrate-binding domains within the DNA enzyme are underlined).

For A
additional nucleotides
(SEQ ID NO: 28)
5′-GAGACCGCAAGACCCCCCAG-3′,
guide RNA
(SEQ ID NO: 29)
5′-GGUCUUGCGGUCUCACCA-3′;
For A′
additional nucleotides
(SEQ ID NO: 30)
5′-GAGACCGCAUCUGAGACGAUGU-3′,
guide RNA
(SEQ ID NO: 31)
5′-GGCAGAUGCGGUCUCACCA-3′;
For B
additional nucleotides
(SEQ ID NO: 32)
5′-AGACCCCCCAG•UACACACACC-3′,
DNA enzyme
(SEQ ID NO: 33)
5′-GGTGTGTGTAGGCTAGCTACAACGATGGGGGGTCT-3′;
For B′
additional nucleotides
(SEQ ID NO: 34)
5′-UCUGAGACGAUG•UUGAAAAGAGAG-3′,
DNA enzyme
(SEQ ID NO: 35)
5′-CTCTCTTTTCAAGGCTAGCTACAACG AATCGTCTCAGT-3′.

DNA-catalyzed cleavage is carried out in a reaction mixture containing 10 μM RNA, 30 μM DNA enzyme, 25 mM CaCl₂, and 30 mM EPPS (pH 7.5), which is heated to 70° C. for 2 minutes, then incubated at 37° C. for 45 minutes. Following RNA- or DNA-catalyzed cleavage, the desired products are purified by PAGE.

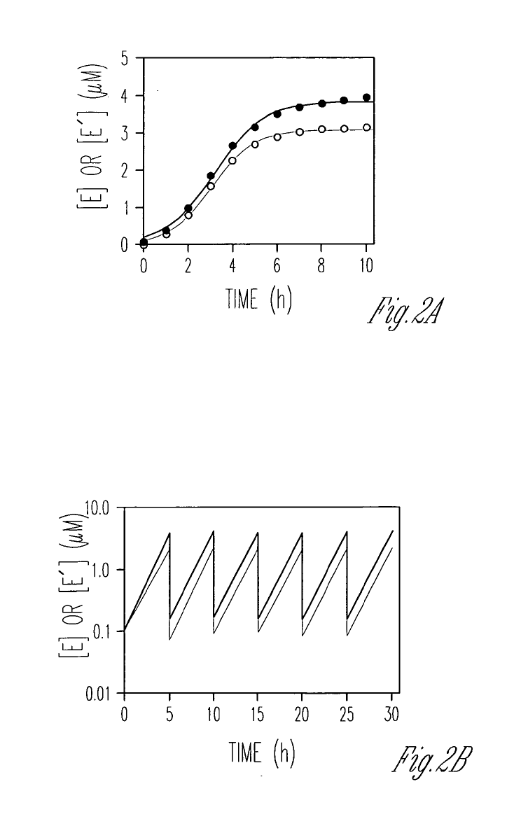

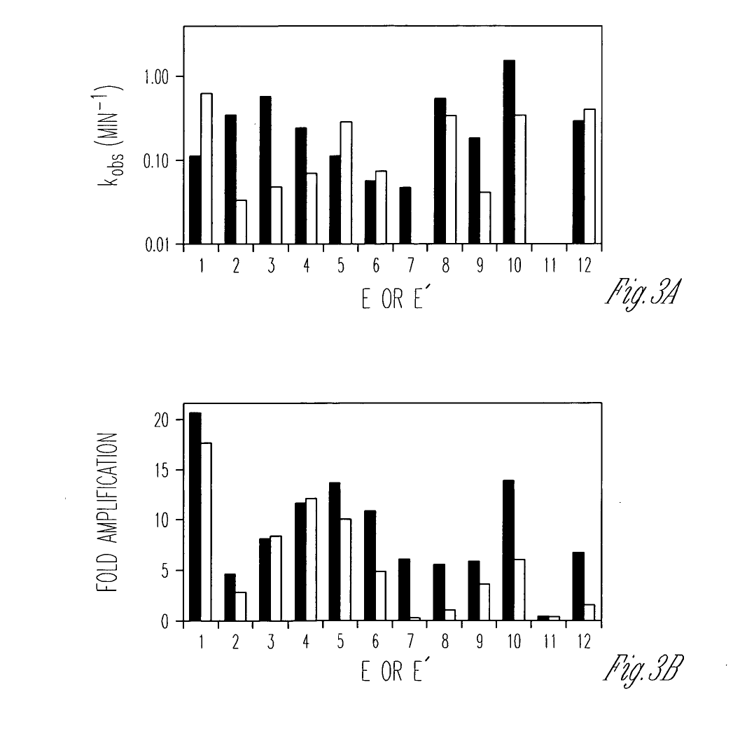

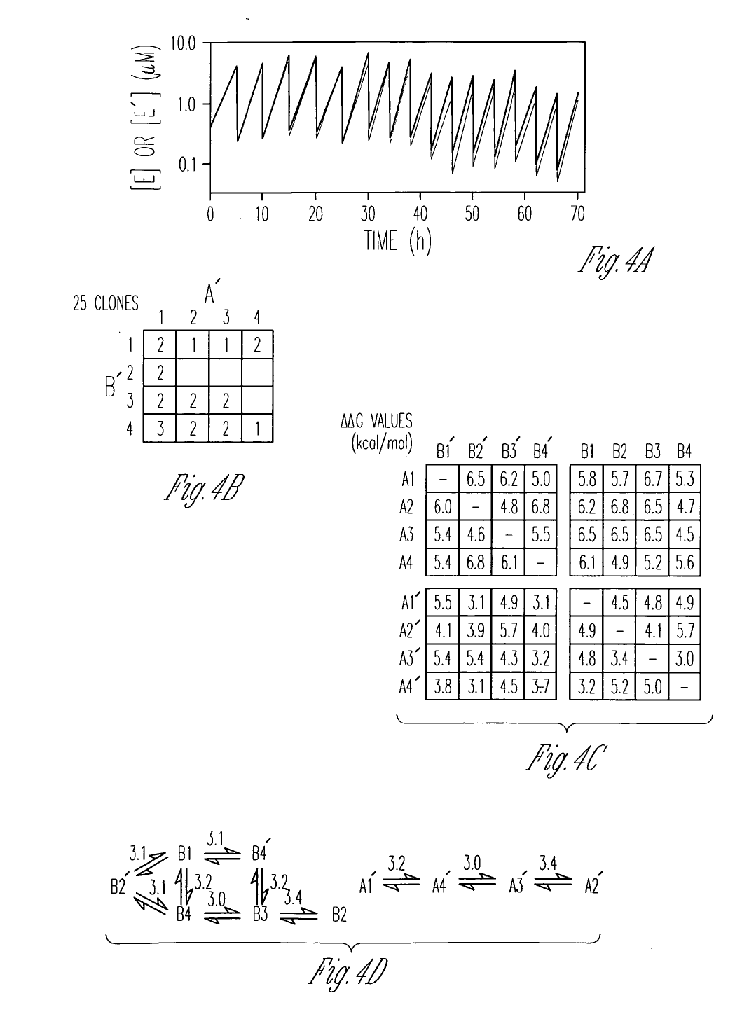

Serial transfer experiments. Reaction mixtures for exponential amplification of cross-replicating RNAs contained 5 μM each of the A, A′, B, and B′ substrates, 15 or 25 mM MgCl₂, and 50 mM EPPS (pH 8.5), which were incubated at 42° C. The first reaction mixture in a serial transfer experiment contained 0.1 μM each of E and E′, but all subsequent mixtures contained only the E and E′ molecules that were carried over in the transfer. When multiple cross-replicating RNAs were employed, each was present at 0.1 μM concentration in the first reaction mixture, and 5 μM each of the component substrates were present in all of the reaction mixtures. The experiment involving E1 and E1′ alone (FIG. 2B) was carried out in the presence of 25 mM MgCl₂, while the experiments involving multiple pairs of cross-replicating enzymes (FIGS. 3A and 4 a) were carried out in the presence of 15 mM MgCl₂.

The experiment involving 12 pairs of cross-replicating enzymes was pre-initiated by amplifying each cross-replicator in isolation for 10 hours, determining the concentrations of E and E that had been produced, and employing an aliquot from these mixtures containing a total of 0.2 μM enzymes to initiate the first reaction of the serial transfer procedure. The enzymes E11 and E11′ amplified so poorly that in their case 0.1 μM of each enzyme was employed directly. The pre-initiation procedure was carried out so that the first reaction of the serial transfer would more closely resemble subsequent reactions with regard to the relative amounts of the two members of a cross-replicating pair (FIG. 3B). The enzyme E12′ formed a (5′-UAUG-3′)•(5′-AUAC-3′) mismatch with the A12 substrate, but there was no mismatch between E12 and B12′.

In order to prepare the products of a serial transfer experiment for cloning and sequencing, the E and E′ molecules were purified by PAGE, then 3′-polyadenylated, reverse transcribed, and tailed at the 3′ end of the cDNA using terminal transferase. The polyadenylation reactions contained about 0.4 μM E (or E′), 0.1 U/μL poly(A) polymerase, 0.5 mM ATP, 10 mM MgCl₂, 250 mM NaCl, and 50 mM Tris-HCl (pH 8.0), which was incubated for 2 hours at 37° C. The polymerase was extracted with phenol/chloroform, the mixture was desalted using a NAP column (GE Healthcare, Piscataway, N.J.), and the extended RNAs were reverse transcribed as described above, using a DNA primer having the sequence 5′-T₂₄N-3′ (N=A, C or G). Full-length cDNAs were purified by PAGE, then extended in a reaction mixture containing about 0.2 μM cDNA, 8 U/μL terminal transferase, 1 mM dGTP, 2.5 mM CoCl₂, 200 mM potassium cacodylate, 0.25 mg/ml BSA, and 25 mM Tris-HCl (pH 6.6), which was incubated at 37° C. for 2 hours. The proteins were extracted with phenol/chloroform, the mixture was desalted using a NAP column, and the extended cDNAs were PCR amplified using primers having the sequence 5′-GACAGATCAGT₂₄N-3′ (SEQ ID NO:37; N=A, C or G) and 5′-GGCTAACACGAC₁₄G-3′ (SEQ ID NO:38). The PCR products were cloned and sequenced.

Kinetic analysis. RNA-catalyzed RNA ligation was carried out in a reaction mixture containing 5 μM E (or E′), 0.1 μM [5′-³²P]-labeled A′ (or A), 6 μM B′ (or B), 15 or 25 mM MgCl₂, and 50 mM EPPS (pH 8.5), which was incubated at 30° C. The reaction was initiated by mixing equal volumes of two solutions, one containing the enzymes and substrates, and the other containing the MgCl₂ and EPPS buffer. Aliquots were taken at various times and quenched by adding an equal volume of gel-loading buffer containing 25 mM Na₂EDTA and 18 M urea. The products were separated by PAGE and quantitated using a PharosFX molecular imager (Bio-Rad, Hercules, Calif.). The data were fit to the equation:

Fₜ=a(1−e^{-kt})+b,
where F^{t} is the fraction reacted at time t, a is the maximum extent of the reaction (typically 0.88-0.92), k is the observed rate of product formation, and b is the calculated extent at t=0 (typically 0.01-0.03).
   Reactions catalyzed by E7′, E11, and E11′ were so slow that the data instead were fit to the linear equation: Fₜ=at+b.

Cross-catalytic exponential amplification was carried out in a reaction mixture containing 0.1 μM each of E and E′, 5 μM each of [5′-³²P]-labeled A and A′, 5 μM each of B and B′, 15 or 25 mM MgCl₂, and 50 mM EPPS (pH 8.5), which was incubated at 42° C. The reaction was initiated as described above. Aliquots were taken at various times, quenched, and the amounts of newly-synthesized E and E′ were quantitated as described above. The data were fit to the logistic growth equation, as described in the main text. This equation is commonly used in population ecology to model the exponential growth of organisms subject to the carrying capacity of the local environment.

### Results

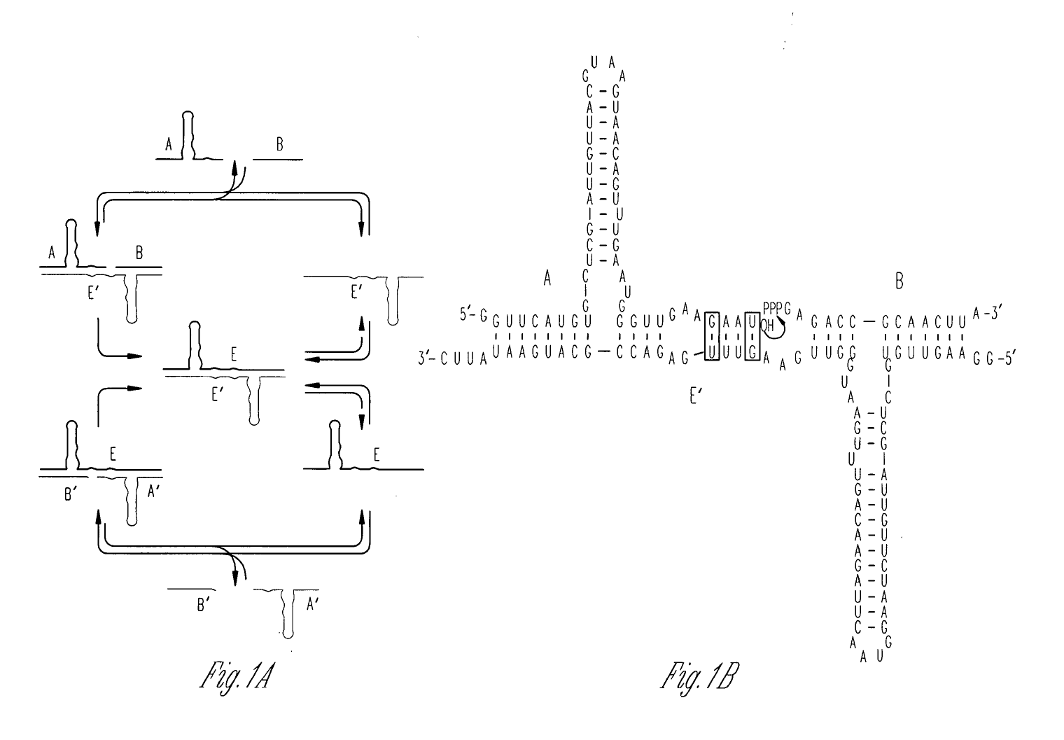

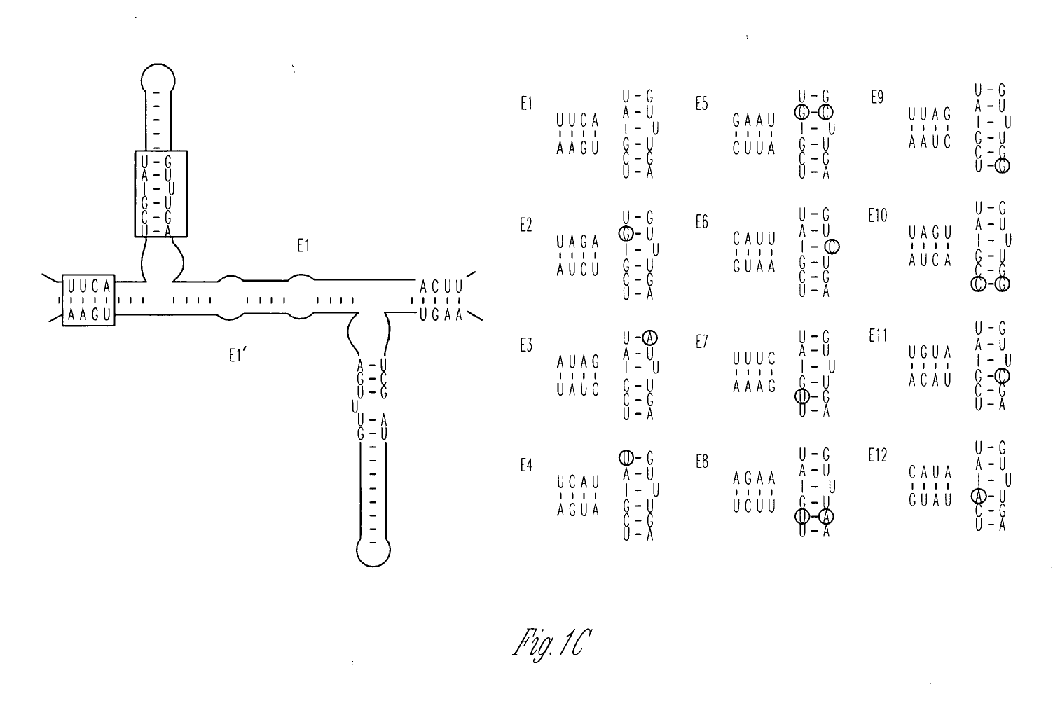

The R3C ligase was converted to a cross-catalytic format (FIG. 1A), whereby a plus-strand RNA enzyme (E) catalyzes the joining of two substrates (A′ and B′) to form a minus-strand enzyme (E′), which in turn catalyzes the joining of two substrates (A and B) to form a new plus-strand enzyme (Kim and Joyce, 2004; Kim et al., 2008). This too was inefficient because of the formation of non-productive complexes and the slow underlying rate of the two enzymes. The enzymes E and E′ operate with a rate constant of only about 0.03 minute⁻¹ and a maximum extent of only 10-20% (Kim and Joyce, 2004). These rates are about 10-fold slower than that of the parental R3C ligase (Rogers and Joyce, 2001), and when the two cross-catalytic reactions are carried out within a common mixture, the rates are even slower (Kim and Joyce, 2008).

The catalytic properties of the cross-replicating RNA enzymes were improved using in vitro evolution, optimizing the two component reactions in parallel and seeking solutions that would apply to both reactions when conducted in the cross-catalytic format (Kim and Joyce, 2004). The 5′-triphosphate bearing substrate was joined to the enzyme via a hairpin loop (B′ to E, and B to E′), and nucleotides within both the enzyme and the separate 3′-hydroxyl-bearing substrate (A′ and A) were randomized at a frequency of 12% per position. The two resulting populations of molecules were subjected to six rounds of stringent in vitro selection, selecting for their ability to react in progressively shorter times, ranging from 2 hours to 10 milliseconds. Mutagenic PCR was performed after the third round to maintain diversity in the population. Following the sixth round, individuals were cloned from both populations and sequenced. There was substantial sequence variability among the clones, but all contained mutations just upstream from the ligation junction that resulted in a G•U wobble pair at this position.

The G•U pair was installed in both enzymes and both 3′-hydroxyl-bearing substrates (FIG. 1B). In the trimolecular reaction (with two separate substrates), the optimized enzymes, E and E′, exhibited a rate constant of 1.3 and 0.3 minute⁻¹ with a maximum extent of 92% and 88%, respectively. The optimized enzymes underwent robust exponential amplification at a constant temperature of 42° C., with more than 25-fold amplification after 5 hours, followed by a leveling off as the supply of substrates became depleted (FIG. 2A). The data fit well to the logistic growth equation: [E]ₜ=a/(1+be−ct), where [E]ₜ is the concentration of E (or E′) at time t, a is the maximum extent of growth, b is the degree of sigmoidicity, and c is the exponential growth rate. For the enzymes E and E′, the exponential growth rate was 0.92 and 1.05 hour⁻¹, respectively.

Exponential growth can be continued indefinitely in a serial transfer experiment in which a portion of a completed reaction mixture is transferred to a new reaction vessel that contains a fresh supply of substrates. Six successive reactions were carried out in this fashion, each 5 hours in duration and transferring 1/25th of the material from one reaction mixture to the next. The first mixture contained 0.1 μM each of E and E′, but all subsequent mixtures contained only those enzymes that were carried over in the transfer. Exponential growth was maintained throughout 30 hours total incubation, with an overall amplification of >10⁸-fold for each of the two enzymes (FIG. 2B). This corresponds to 28 doublings in a process that was sustained by the enzymes themselves. No temperature cycling was required and the reaction mixtures did not contain any proteins or other biological materials.

A genetic system requires not only self-replication, but also the opportunity for many different genetic molecules to replicate, with their replication rate dependent on genetically-encoded functional properties. It is possible to construct many variants of the cross-replicating RNA enzymes that differ with respect to their “genotype” and associated “phenotype”. The genotype is defined as the regions of the enzyme that engage in Watson-Crick pairing with its cross-catalytic partner and that can vary in sequence without significantly affecting replication efficiency. These regions are located at the 5′ and 3′ ends of the enzyme (FIG. 1B). Other regions of Watson-Crick pairing between the two enzymes are tolerant of some sequence variation, albeit with some alteration of replication efficiency.

It is possible to construct variants of the cross-replicating RNA enzymes that differ in the regions of Watson-Crick pairing between the cross-catalytic partners, without markedly affecting replication efficiency. These regions are located at the 5′ and 3′ ends of the enzyme (FIG. 1B). Four nucleotide positions at both the 5′ and 3′ ends were varied, adopting the rule that each region contain one G•C and three A•U pairs so that there would be no substantial differences in base-pairing stability. Of the 32 possible pairs of complementary sequences for each region, 12 were chosen as a set of designated pairings (FIG. 1C). Each genotype was associated with a distinct phenotype, manifest as a particular sequence within the catalytic core of the enzyme. For simplicity, the same phenotype was associated with both members of a cross-replicating pair, although this need not be the case. Each pairing was associated with a particular sequence within the catalytic core of both members of a cross-replicating pair.

Twelve pairs of cross-replicating enzymes were synthesized, as well as the 48 substrates (12 each of A, A′, B, and B′) necessary to support their exponential amplification. Each replicator was tested individually and demonstrated varying levels of catalytic activity and varying rates of exponential growth (FIG. 3A). The pair shown in FIG. 1B (now designated E1 and E1′) had the fastest rate of exponential growth, achieving about 20-fold amplification after 5 hours. The various cross-replicating enzymes shown in FIG. 1C had the following rank order of replication efficiency: E1, E10, E5, E4, E6, E3, E12, E7, E9, E8, E2, E11. The top five replicators all achieved more than 10-fold amplification after 5 hours, and all except E11 achieved at least 5-fold amplification after 5 hours.

A serial transfer experiment was initiated with 0.1 μM each of E1-E4 and E1′-E4′, and 5.0 μM each of the 16 corresponding substrates. Sixteen successive transfers were carried out over 70 hours, transferring 1/20th of the material from one reaction mixture to the next (FIG. 4A). Individuals were cloned from the population following the final reaction and sequenced. Among 25 clones (sequencing E′ only), there was no dominant replicator (FIG. 4B). E1′, E2′, E3′, and E4′ all were represented, as well as 17 clones that were the result of recombination between a particular A′ substrate and one of the three B′ substrates other than its original partner (or similarly for A and B). Recombination occurs when an enzyme binds and ligates a mismatched substrate. In principle, any A could become joined to any B or B′, and any A′ could become joined to any B′ or B, resulting in 64 possible enzymes. The set of replicators were designed so that cognate substrates have a binding advantage of several kcal/mol compared to non-cognate substrates (FIG. 4C), but once a mismatched substrate is bound and ligated, it forms a recombinant enzyme that also can cross-replicate. Recombinants can give rise to other recombinants, as well as revert back to non-recombinants. Based on relative binding affinities, there are expected to be preferred pathways for mutation, primarily involving substitution among certain A′ or among certain B components (FIG. 4D).

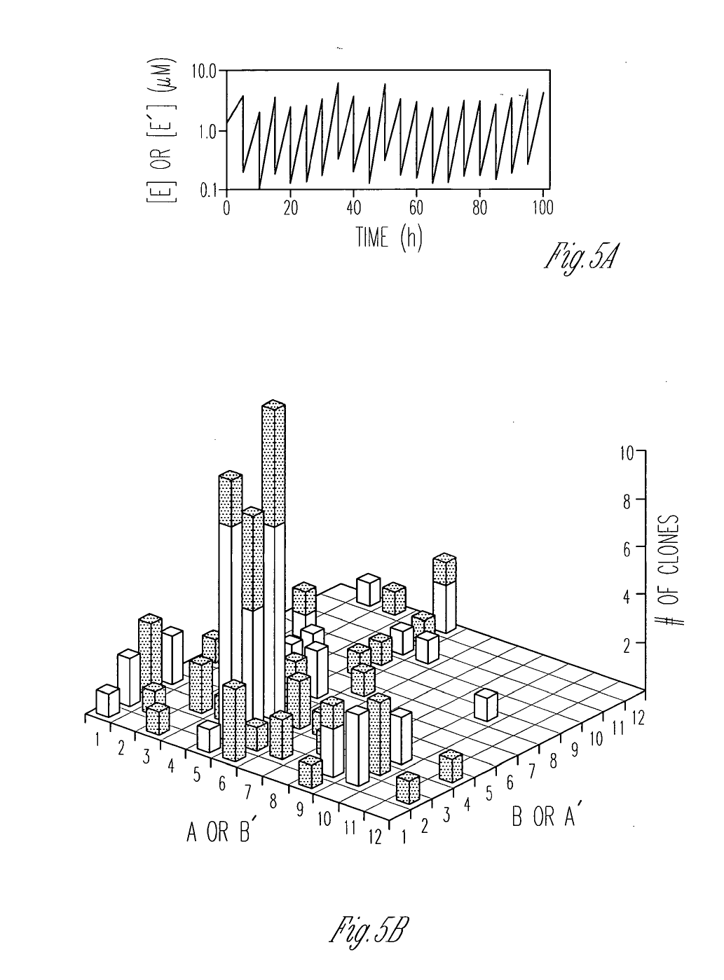

A second serial transfer experiment was initiated with 0.1 μM each of all 12 pairs of cross-replicating enzymes and 5.0 μM each of the 48 corresponding substrates. This mixture allowed 132 possible pairs of recombinant cross-replicating enzymes, as well as the 12 pairs of non-recombinant cross-replicators. Twenty successive reactions were carried out over 100 hours, transferring 1/20th of the material from one reaction mixture to the next, and achieving an overall amplification of >1025-fold (FIG. 5A). Of 100 clones isolated following the final reaction (sequencing 50 E and 50 E′), only 7 were non-recombinants (FIG. 5B). The distribution was highly non-uniform, with sparse representation of molecules containing components A6-A12 and B5-B12 (and reciprocal components B6′-B12′ and A5′-A12′). The most frequently represented components were A5 and B3 (and reciprocal components B5′ and A3′). The three most abundant recombinants were A5B2, A5B3, and A5B4 (and their cross-replication partners), which together accounted for one-third of all clones.

In the presence of their cognate substrates alone, E1 remained the most efficient replicator, but in the presence of all 48 substrates, the most efficient replicator was A5B3 (FIG. 5C). When the A5B3 replicator was provided a mixture of substrates corresponding to the components of the three most abundant recombinants, its exponential growth rate was the highest measured for any replicator (FIG. 5D). The fitness of a pair of cross-replicating enzymes depends on several factors, including their intrinsic catalytic activity, exponential growth rate with cognate substrates, ability to withstand inhibition by other substrates in the mixture, and net rate of production through mutation among the various cross-replicators. The A5B3 recombinant and its cross-replication partner B5′A3′ have different catalytic cores (FIG. 1C), and both exhibit comparable activity, accounting for their well-balanced rate of production throughout the course of exponential amplification (FIG. 5D). The selective advantage of this cross-replicator appears to derive from its relative resistance to inhibition by other substrates in the mixture (FIG. 5C) and its ability to capitalize on facile mutation among substrates B2, B3, and B4 and among substrates A2′, A3′, and A4′ that comprise the most abundant recombinants (FIG. 5D).

Populations of cross-replicating RNA enzymes can serve as a simplified experimental model of a genetic system with, at present, two genetic loci and 12 alleles per locus. It is likely, however, that the number of alleles could be increased by exploiting more than four nucleotide positions at the 5′ and 3′ ends of the enzyme, and by relaxing the rule that these nucleotides form one G•C and three A•U pairs. In order to support much greater complexity it will be necessary to constrain the set of substrates, for example, by using the population of newly-formed enzymes to generate a daughter population of substrates (Kim and Joyce, 2004). An important challenge for an artificial RNA-based genetic system is to support a broad range of encoded functions, well beyond replication itself. Ultimately the system should provide open-ended opportunities for discovering novel function, something that likely has not occurred on Earth since the time of the RNA world, but presents an increasingly tangible research opportunity.

In order to support greater complexity in a system of cross-replicating RNAs it will be necessary to constrain the set of substrates so that each enzyme can secure its own substrates without being overwhelmed by other substrates in the mixture. One way to do this is to choose a set of substrates that are more distinguishable than the ones used here. Another approach is to adjust the concentrations of the various substrates in proportion to their utilization by the population of enzymes. It is not clear how this would be done within the system, but it could be achieved using a deconstructive PCR procedure in which the population of newly-formed enzymes is used to generate a corresponding population of substrates (see Example 2). In this way both the successful enzymes and their component substrates are inherited from one generation to the next.

Another important challenge for an artificial genetic system is to support a broad range of encoded functions, well beyond replication itself. It is possible to insert a functional domain within the central stem-loop of the cross-replicating enzymes so that replication is dependent on execution of that encoded function (Lam & Joyce, unpublished results). It would be much more powerful, however, to have a system in which novel function emerges during the course of selective amplification. The self-sustained evolution of RNA with open-ended opportunities for discovering novel function likely has not occurred on Earth since the time of the RNA world, and continues to present an intriguing research opportunity.

### Example 2

Catalytic properties of the starting and evolved enzymes. In the trimolecular reaction (with two separate substrates), the parental R3C ligase operates with a k_{cat} of 0.2 min⁻¹, Kₘ of 0.4 μM for the 3′-hydroxyl-terminated substrate, and Kₘ of 0.1 μM for the 5′-triphosphate-terminated substrate, measured in the presence of 25 mM MgCl₂ at pH 8.5 and 23° C. (Rogers et al., 2007). This molecule was converted to an autocatalytic format that enabled limited self-replication (Paul et al., 2002). For the self-replicating enzyme, the substrates A and B have substantial complementarity, resulting in formation of a non-productive A•B complex. This complex was observed by gel-shift studies employing non-denaturing polyacrylamide gels (Paul et al., 2002). Formation of the non-productive complex gives rise to biphasic kinetics, with an initial fast phase of exponential amplification, followed by a slow phase of linear growth. The amplitude of the exponential phase can be increased by increasing the concentration of A relative to B, or by controlling the order of addition, such that A is added to a mixture already containing B and E (Paul et al., 2002).

Gel-shift analysis revealed that for E concentrations of 0.1-100 μM, most of the enzyme molecules exist as a monomer, rather than an E•E dimer or higher-order complex (measured in the presence of 10 mM MgCl₂ at pH 8.5 and 23° C.) (Paul et al., 2002). The availability of free E enables exponential growth with product turnover until the supply of free substrates is exhausted. The behavior of the self-replicating enzyme during the exponential phase can be described by the equation:

(d[E]/dt)ᵢₙᵢₜᵢₐₗ=kₐ[E₀]^{p}+k_{b},
where [E₀] is the starting concentration of E, kₐ is the autocatalytic (E-dependent) rate constant, k_{b} is the non-autocatalytic (E-independent) rate constant, and p is the reaction order.
   In the presence of 2 μM each of A and B and 25 mM MgCl₂ at pH 8.5 and 23° C., kₐ=0.011 min⁻¹, k_{b}=3.3×10⁻¹¹ M·min⁻¹, and p=1.0. Under these conditions, the amplitude of the exponential phase is about 5% (Paul et al., 2002).

The original cross-replicating enzyme has nearly identical sequence compared to the self-replicating enzyme, except for five altered nucleotides in the pairing regions at the 5′ and 3′ ends, and three base pairs added to the central stem to provide a size difference between E and E′ (Kim et al., 2004). In the trimolecular reaction, the original E operates with a rate constant of 0.034 min⁻¹ and amplitude of 20% in the fast phase, followed by a slow phase with a rate of 5.0×10⁻⁴ min⁻¹, while E′ operates with a rate constant of 0.026 min⁻¹ and amplitude of 11% in the fast phase, followed by a slow phase with a rate of 4.0×10⁻⁴ min⁻¹ (measured in the presence of 1 μM E or E′, 2 μM A′ or A, 2 μM B′ or B, and 25 mM MgCl₂ at pH 8.5 and 23° C.).

Pulse-chase experiments were carried out to determine the dissociation rate of the E•E′ complex at various temperatures, revealing a rate of 0.09 min⁻¹ at 23° C., 0.14 min⁻¹ at 33° C., and 0.18 min⁻¹ at 43° C. (Kim et al., 2004). These rates are faster than the rate constant for the individual RNA-catalyzed ligation reactions. When the reactions catalyzed by E and E′ are carried out in a common reaction mixture (employing 1 μM each of E and E′, and 2 μM each of A′, A, B′, and B), E has a rate constant of 6.1×10⁻³ min⁻¹ and amplitude of 15% in the fast phase, followed by a slow phase with a rate of 5.4×10⁻⁵ min⁻¹, while E′ has a rate constant of 6.2×10⁻³ min⁻¹ and amplitude of 8% in the fast phase, followed by a slow phase with a rate of 5.1×10⁻⁵ min⁻¹ (Kim et al., 2004).

Kim and colleagues (Kim et al., 2008) carried out temperature cycling experiments using a slightly modified form of the original cross-replicating enzyme that contains an extra G•C pair in each of the two pairing regions. These molecules exhibited similar behavior in the individual RNA-catalyzed reactions compared to the molecules described above. When the two reactions were carried out in a common reaction mixture at a constant temperature of 23° C. (employing 1 μM each of E and E′, 2 μM each of A′, A, B′, and B, and 25 mM MgCl₂ at pH 8.5), the maximum extent was only 1% and 3% for reactions catalyzed by E and E′, respectively. However, this increased to 9% and 13%, respectively, when the temperature was raised to 55° C. every 30 minutes over a total reaction period of 6.5 hours (Kim et al., 2008).

The optimized cross-replicating enzyme obtained in the present study has substantially improved catalytic properties compared to the previous version. Prior to initiating in vitro evolution, the sequence of the central stem (the portion of E that binds the 3′ end of A′, and reciprocally for E′ and A) was changed from (5′-UAUA-3′)•(5′-UAUA-3′) to (5′-UAAA-3′)•(5′-UUUA-3′). This change was made to disrupt the palindrome of the central stem in an effort to reduce formation of non-productive complexes. It improved the maximum extent of reaction to 60% and 15% for E and E′, respectively. The maximum extent could not be significantly improved by increasing the concentration of enzyme, suggesting that there is an inherent limitation in one or more of the substrates.

The four substrates were evaluated individually by allowing the reaction to proceed to maximum extent in the presence of 1 to 3 μM enzyme, 1 to 3 nM of the substrate being tested, 1 to 3 μM of the partner substrate, and 25 mM MgCl₂, incubating at pH 8.5 and 30° C. for 24 hours. The tested substrate molecules that did not react were purified by PAGE and used in a second RNA-catalyzed reaction. The maximum extents of the two successive reactions were as follows:

| Substrate | 1st reaction | 2nd reaction | Total extent |
|---|---|---|---|
| A′ | 71% | 0% | 71% |
| B′ | 48% | 3% | 51% |
| A | 51% | 17% | 68% |
| B | 44% | 6% | 50% |

This indicated that a substantial fraction of the substrates have compositional defects, as well as conformational defects in the case of the A molecules. Accordingly, A and A′ were prepared as extended length transcripts and cleaved using E. coli M1 RNA to generate precise 3′ termini. This improved the maximum extent of reaction to about 90%.

The k_{cat} and Kₘ were determined for each of the four substrates in the presence of a saturating concentration of their partner substrate and 25 mM MgCl₂ at pH 8.5 and 30° C. Reactions were performed using various concentrations of E or E′ and trace amounts of the substrate being evaluated. The data fit well to the Michaelis-Menten equation, which was used to obtain the following catalytic parameters:

| Substrate | k_{cat}(min⁻¹) | kₘ(μM) |
|---|---|---|
| A′ | 0.03 | 0.3 |
| B′ | 0.01 | 0.03 |
| A | 0.02 | 0.5 |
| B | 0.03 | 0.06 |

In vitro evolution was carried out as described above, resulting in optimized cross-replicating enzymes with the critical wobble pairs in the central stem (FIG. 1B). In order to achieve a high maximum extent, it still was necessary to employ M1 RNA to prepare A and A′ molecules with precise 3′ termini. In the trimolecular reaction, the optimized enzyme E operates with a rate constant of 1.3 min⁻¹ and maximum extent of 92%, while E′ operates with a rate constant of 0.3 min⁻¹ and maximum extent of 88%, measured in the presence of 5 μM E or E′, 0.1 μM [5′-³²P]-labeled A′ or A, 6 μM B′ or B, and 25 mM MgCl₂ at pH 8.5 and 30° C. Both reactions exhibit monophasic kinetics. The reactions require Mg²⁺, but the rate constant is unchanged over MgCl₂ concentrations of 5 to 35 mM. The rate constant increases with increasing pH over the range of 6.5 to 9.0, although at pH 9.0 (and especially at 42° C.) the amount of RNA degradation is substantial.

Cross-catalytic replication was carried out with the optimized enzymes, comparing reactions performed in the presence of either 15 or 25 mM MgCl₂ and at either 30 or 42° C. (always at pH 8.5). Exponential amplification is approximately two-fold faster in the presence of 25 compared to 15 mM MgCl₂, suggesting that dissociation of the E•E′ complex is not rate limiting. The higher MgCl₂ concentration was adopted for the initial test of the E1 replicator (FIG. 2), but the lower concentration was used in all subsequent experiments in order to reduce the use of mismatched substrates in mixtures of multiple cross-replicators and to render the RNA less susceptible to hydrolysis. Amplification is about four-fold faster at 42 compared to 30° C. An initial serial transfer experiment was performed at 30° C., involving six successive reactions of 16 hour duration and transferring 1/25th of the material from one reaction mixture to the next (data not shown). However, the same amount of amplification could be achieved in about 4 hours at 42° C., so the higher temperature was used in all subsequent experiments.

Cross-Replicators with Swapped Pairing Domains.

The choice of sequence within the paired regions of the 12 cross-replicating RNA enzymes was arbitrarily related to a particular sequence within the corresponding catalytic core. For simplicity, the same catalytic core sequence was associated with both members of a cross-replicating pair, although this need not be the case. Also arbitrarily, the pairing sequences at the 5′ and 3′ ends of each enzyme were chosen to be identical when one is read in the 5′→3′ and the other in the 3′→5′ direction. This is a convenient way to ensure that the two ends are not complementary.

Variant forms of the E1, E1′, E4, and E4′ enzymes were prepared in which the paired regions within E1 and E1′ were exchanged for those within E4 and E4′, respectively. This was done to assess the independent contributions of the pairing regions and catalytic core to the behavior of the enzyme. For the original and swapped versions of each enzyme, the rate constant was determined in the trimolecular reaction, measured in the presence of 5 μM E or E′, 0.1 μM [5′-³²P]-labeled A′ or A, 6 μM B′ or B, and 15 mM MgCl₂ at pH 8.5 and 30° C. In addition, the exponential growth rate and fold amplification after 5 hours was determined for each pair of cross replicators, measured in the presence of 0.1 μM each of E and E′, 5 μM each of A′, A, B′ and B, and 15 mM MgCl₂ at pH 8.5 and 42° C. These data are summarized below.

The E1 and E4 enzymes have a similar catalytic rate constant, and swapping their catalytic cores had little effect on their behavior in the individual RNA-catalyzed reactions. The E1′ and E4′ enzymes have more disparate rate constants, with E1′ being much faster than E1, and E4′ being much slower than E4. Thus swapping the catalytic cores of E1′ and E4′ reduced activity of the former and increased activity of the latter. Exponential amplification depends on the reciprocal activity of both members of a cross-replicating pair. All of the enzymes exhibited robust exponential amplification, with the E1 and E1′ pair performing best regardless of the choice of catalytic core, and the E4 and E4′ pair performing worst when fitted with the catalytic core originally associated with E1 and E1′.

| | Catalytic | | | |
|---|---|---|---|---|
| Pairing | core | k_{obs} (min⁻¹) | Growth (h⁻¹) | Amplification |
| E1 | E1 | 0.11 | 0.08 | 20.7 |
| E1′ | E1′ | 0.63 | 0.77 | 17.7 |
| E1 | E4 | 0.13 | 0.78 | 22.8 |
| E1′ | E4′ | 0.34 | 0.77 | 12.3 |
| E4 | E1 | 0.24 | 0.42 | 7.1 |
| E4′ | E1′ | 0.22 | 0.48 | 3.3 |
| E4 | E4 | 0.24 | 0.95 | 11.7 |
| E4′ | E4′ | 0.07 | 0.98 | 12.2 |

### Example 3

Materials. Oligonucleotides were synthesized on an Expedite automated DNA/RNA synthesizer (Applied Biosystems, Foster City, Calif.) using nucleoside phosphoramidites purchased from Glen Research (Sterling, Va.). All oligonucleotides were purified by denaturing polyacrylamide gel electrophoresis (PAGE) and desalted using a C18 SEP-Pak cartridge (Waters, Milford, Mass.). Histidine-tagged T7 RNA polymerase was purified from E. coli strain BL21 containing plasmid pBH161 (kindly provided by William McAllister, State University of New York, Brooklyn). Thermus aquaticus DNA polymerase was cloned from total genomic DNA and purified as described in Pluthero (1993). M1 RNA, the catalytic subunit of RNAse P, was obtained from E. coli genomic DNA (Sigma-Aldrich, St. Louis, Mo.) by PCR amplification and subsequent in vitro transcription, as described in Example 1. Calf intestine phosphatase and T4 polynucleotide kinase were purchased from New England Biolabs (Ipswich, Mass.), yeast inorganic pyrophosphatase was from Sigma-Aldrich, and bovine pancreatic DNase I was from Roche Applied Science (Indianapolis, Ind.). Nucleoside and deoxynucleoside 5′-triphosphates, theophylline, and FMN were purchased from Sigma-Aldrich, [γ-³²P]ATP (7 μCi/pmol) was from Perkin Elmer (Waltham, Mass.), and caffeine was from MP Biomedicals (Solon, Ohio). Photinus pyralis (firefly) luciferase, Saccharomyces cerevisiae adenosine-5′-triphosphate sulfurylase, adenosine 5′-phosphosulfate, and D-luciferin were from Sigma-Aldrich.

Preparation of aptazymes and substrates. All RNA enzymes and substrates were prepared by in vitro transcription in a reaction mixture containing 0.4 μM DNA template, 0.8 μM synthetic oligodeoxynucleotide having the sequence 5′-GGACTAATACGACTCACTATA-3′ (SEQ ID NO:39) (T7 RNA polymerase promoter sequence underlined), 2 mM each of the four NTPs, 15 U/μL T7 RNA polymerase, 0.001 U/μL inorganic pyrophosphatase, 15 mM MgCl₂, 2 mM spermidine, 5 mM dithiothreitol, and 50 mM Tris-HCl (pH 7.5). The mixture was incubated at 37° C. for 2 hours, quenched by adding an equal volume of 15 mM Na₂EDTA, treated with 1 U/μL DNase I, and extracted with a 1:1 mixture of phenol:chloroform. The RNA was precipitated, purified by PAGE, and desalted. Transcription of M1 RNA was performed similarly, except employing a double-stranded DNA template that was generated by PCR.

The A and A′ substrates could not be obtained reliably by in vitro transcription due to heterogeneity at the 3′ end of the transcripts. Instead, these substrates were prepared from the corresponding E or E′ molecules by cleaving off the B or B′ portion using E. coli M1 RNA, as described in Example 1. The external guide sequence RNA (Forster and Altmann, 1990) for cleavage of Eₜₕₑₒ and E_{FMN} had the sequence 5′-CGUAAGUUGCGGUCUCACCA-3′ (SEQ ID NO:40), and for E′ₜₕₑₒ and E′_{FMN} had the sequence 5′-AUAUUCAUGCGGUCUCACCA-3′ (SEQ ID NO:41) (nucleotides complementary to the target RNA underlined). For the second pair of Eₜₕₑₒ and E′ₜₕₑₒ molecules used in the multiplex experiments, the external guide sequence RNAs had the sequence 5′-CGUAGUAUGCGGUCUACCA-3′ (SEQ ID NO:42) and 5′-GAAUAUCAUUGCGGUCUCACCA-3′ (SEQ ID NO:43), respectively. The A and A′ substrates were [5′-³²P]-labeled by first dephosphorylating using calf intestine alkaline phosphatase, then phosphorylating using T4 polynucleotide kinase and [γ-³²P]ATP. The labeled substrates were purified by PAGE and desalted using a Nensorb 20 cartridge (NEN Life Sciences, Waltham, Mass.).

Individual RNA-catalyzed reactions. RNA-catalyzed RNA ligation was performed in a reaction mixture containing 5 μM E or E′, 0.1 μM [5′-³²P]-labeled A′ or A, 6 μM B′ or B, 25 mM MgCl₂, and 50 mM EPPS (pH 8.5), which was incubated at 42° C. Aliquots were taken at various times and quenched by adding an equal volume of gel-loading buffer containing 50 mM Na₂EDTA and 18 M urea. The products were separated by PAGE and quantitated using a PharosFX molecular imager (Bio-Rad, Hercules, Calif.). The data were fit to the equation:

Fₜ=Fₘₐₓ−(a1e^{k1·t})−(a2e^{k2·t}),
where Fₜ is the fraction reacted at time t, Fₘₐₓ is the overall maximum extent of the reaction, a1 and k1 are the amplitude and rate of the initial fast phase, and a2 and k2 are the amplitude and rate of the subsequent slow phase, respectively.
   In the presence of 5 mM theophylline, the reaction catalyzed by Eₜₕₑₒ exhibited a fast phase with an amplitude of 0.57 and rate of 1.4 minutes⁻¹, followed by a slow phase with an amplitude of 0.24 and rate of 0.044 minutes⁻¹; the reaction catalyzed by E′ₜₕₑₒ had an amplitude of 0.52 and rate of 0.59 minutes⁻¹ in the fast phase, and an amplitude of 0.26 and rate of 0.045 minutes⁻¹ in the slow phase.

Cross-replication reactions. Cross-catalytic exponential amplification was performed in a reaction mixture containing 0.02 μM each of E and E′, 5 μM each of [5′-³²P]-labeled A and A′, 5 μM each of B and B′, 25 mM MgCl₂, and 50 mM EPPS (pH 8.5), which was incubated at 42° C. The reaction was initiated by mixing equal volumes of two solutions, one containing the enzymes and substrates, and the other containing the MgCl₂ and EPPS buffer. Aliquots were taken at various times, quenched, and the amounts of newly-synthesized E and E′ were quantitated as described above. The data were fit to the logistic growth equation.

Luciferase assays. Known concentrations of inorganic pyrophosphate or samples taken from the cross-replication reaction were diluted 10-fold into a reaction mixture containing 0.15 μg/μL luciferase, 0.00045 U/μL ATP sulfurylase, 10 μM adenosine 5′-phosphosulfate, 0.5 mM D-luciferin, 25 mM magnesium acetate, 0.1% bovine serum albumin, 1 mM dithiothreitol, 0.4 μg/μL polyvinylpyrrolidone (MW 360,000), and 100 mM Tris-acetate (pH 7.75). The pyrophosphate standards were prepared in a solution identical to that employed in cross-replication, but lacking the RNA enzymes and substrates. Luminescence was detected using a Perkin Elmer LS55 luminescence spectrometer operating in bioluminescence mode, with a PMT voltage of 900 volts, cycle time of 200 milliseconds, gate time of 180 milliseconds, and delay time of 0. The flash count was set to 1, the emission filter was fully open, and the emission slit width was 12 nanometers. Following addition of the sample to the luciferase mixture, luminescence was monitored for 5 minutes with a 0.1 second integration time. The amount of light generated was linear over a pyrophosphate concentration range of 0.1-10 μM.

### Results

RNA enzymes have been developed that undergo self-sustained replication at a constant temperature in the absence of proteins (Example 1). These RNA molecules amplify exponentially through a cross-replicative process, whereby two enzymes catalyze each other's synthesis by joining component oligonucleotides. Other RNA enzymes have been made to operate in a ligand-dependent manner by combining a catalytic domain with a ligand-binding domain (aptamer) to provide an “aptazyme” (Tang and Breaker, 1997; Seetharaman et al., 2001; Hesselberth et al., 2003). The principle of ligand-dependent RNA catalysis now has been extended to the cross-replicating RNA enzymes so that exponential amplification occurs in the presence, but not the absence, of the cognate ligand. The exponential growth rate of the RNA depends on the concentration of the ligand, enabling one to determine the concentration of ligand in a sample. This process is analogous to quantitative PCR (qPCR), but can be generalized to a wide variety of targets, including proteins and small molecules that are relevant to medical diagnostics and environmental monitoring.

A well-studied class of RNA enzymes are the RNA ligases, which catalyze the RNA-templated joining of RNA molecules. Some RNA ligases have been made to operate as aptazymes, and some of these have been made to undergo ligand-dependent catalytic turnover to provide linear signal amplification with ongoing target recognition (Hartig et al., 2002; Vaish et al., 2002). One of the RNA ligases is the “R3C” RNA enzyme, which was obtained using in vitro evolution (Rogers and Joyce (2001). This enzyme has been reconfigured so that it can self-replicate by joining two RNA molecules that result in formation of another copy of itself (Paul and Joyce, 2002). It also has been converted to a cross-catalytic format, whereby two RNA enzymes catalyze each other's synthesis from a total of four RNA substrates (Kim and Joyce, 2004). The cross-replication process is analogous to the ligase chain reaction (Wu and Wallace, 1989; Barany, 1991), except that in cross-replication the nucleic acid being amplified is itself the ligase, and strand separation occurs spontaneously without requiring temperature cycling.

The original cross-replicating RNA enzymes were slow catalysts that amplified poorly (Kim and Joyce, 2004). Recently their activity was greatly improved so that they can undergo efficient exponential amplification, generating about a billion copies in 30 hours at a constant temperature of 42° C. (Example 1). Exponential amplification can be continued indefinitely, so long as a supply of the four substrates is maintained. The reaction requires 5-25 mM Mg²⁺, but does not require any proteins or other biological materials.

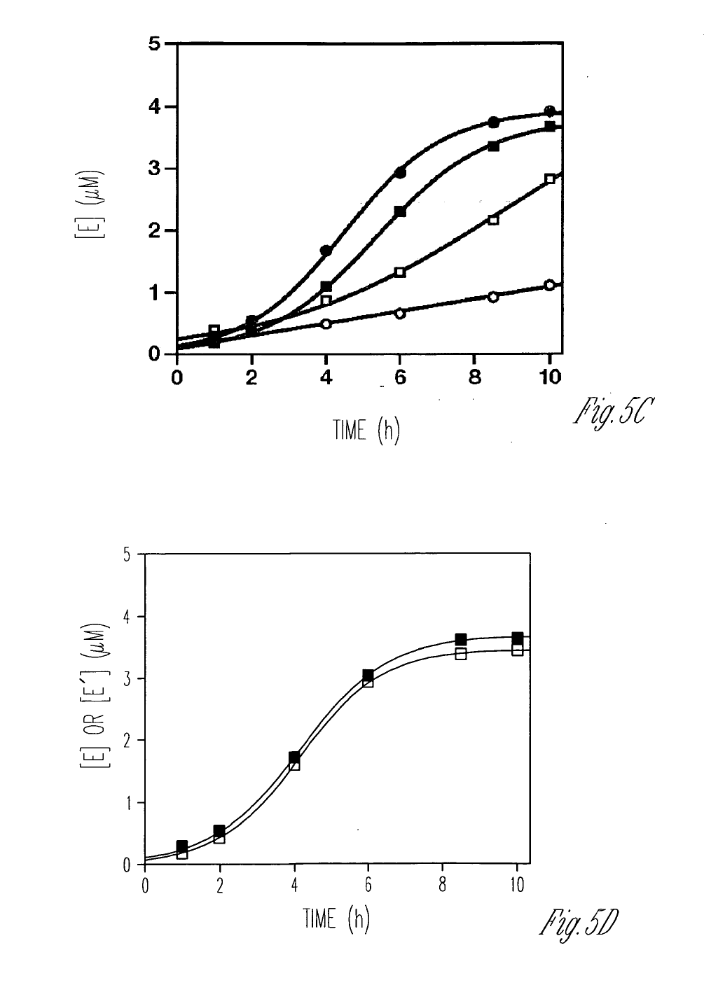

Cross-replication involves a plus-strand RNA enzyme (E) that catalyzes the joining of two substrates (A′ and B′) to form a minus-strand enzyme (E′), which in turn catalyzes the joining of two substrates (A and B) to form a new plus-strand enzyme (E). The cross-replicating enzymes were converted to aptazymes by replacing the distal portion of the central stem-loop by an aptamer that binds a particular ligand (FIG. 6). The ligand binding domain for any ligand may be modified to alter the binding kinetics, e.g., for the theophylline binding domain in FIG. 6, replacing the C/G base pair above G•A with U/AA/U increased the sensitivity of the assay by five-fold (from 0.5 mM to 0.1 mM), likely by increasing stability, without increasing background. The aptamer was installed in the substrates A and A′, and in the corresponding enzymes E and E′. Two different aptamers were chosen, one that binds theophylline (theo) (Jenison et al., 1994) and another that binds flavin mononucleotide (FMN) (Burgstaller and Famulok, 1994). In the absence of the ligand the aptamer domain is unstructured, resulting in destabilization of the adjacent catalytic domain, while in the presence of the ligand the catalytic domain becomes ordered so that exponential amplification can occur. The stability of the stem region connecting the aptamer and catalytic domains was adjusted to maximize the ratio of activity in the “on” (ligand present) compared to “off” (ligand absent) states. Unlike for conventional aptazymes, ligand-dependent activity is expressed exponentially in the growth rate of autocatalytic aptazymes, establishing sharp thresholds for ligand-dependent behavior.

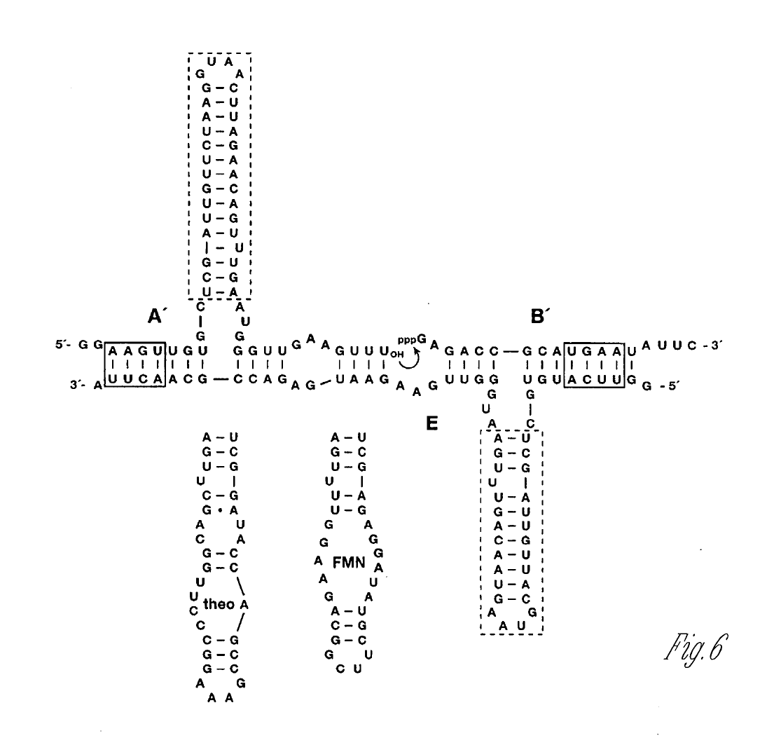

The two theophylline-dependent aptazymes, Etheo and E′theo, first were tested individually in a ligation reaction carried out under saturating conditions in the presence of 5 mM theophylline, exhibiting reaction rates of 1.4 and 0.6 minutes⁻¹, respectively (FIG. 7). Both enzymes had no detectable activity (<10-4 minutes⁻¹) in the absence of theophylline or in the presence of 5 mM caffeine (which differs from theophylline by the presence of a methyl group at the N7 position of caffeine).

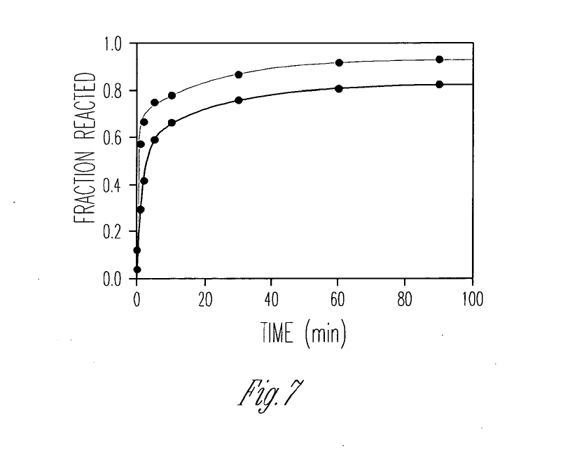

Cross-replication was initiated by adding 0.02 μM each of Etheo and E′theo to a reaction mixture containing 5 μM each of Atheo, A′theo, B, and B′, and either 5 mM theophylline or 5 mM caffeine, which was maintained at a constant temperature of 42° C. Brisk exponential amplification occurred in the presence of theophylline, but there was no detectable amplification in the presence of caffeine (FIG. 8A). Exponential amplification resulted in the formation of new copies of both Etheo and E′theo, ultimately limited by the supply of substrates. A plot of enzyme concentration versus time exhibited a classic sigmoidal shape, indicative of exponential growth subject to a fixed supply of materials. These data were fit to the logistic growth equation:

[E]t=a/(1+be−ct),
where [E]t is the concentration of E (or E′) at time t, a is the maximum extent of growth, b is the degree of sigmoidicity, and c is the exponential growth rate.
   The exponential growth rates of Etheo and E′theo were 0.78 and 0.97 hours⁻¹, respectively, corresponding to a doubling time of about 50 minutes.

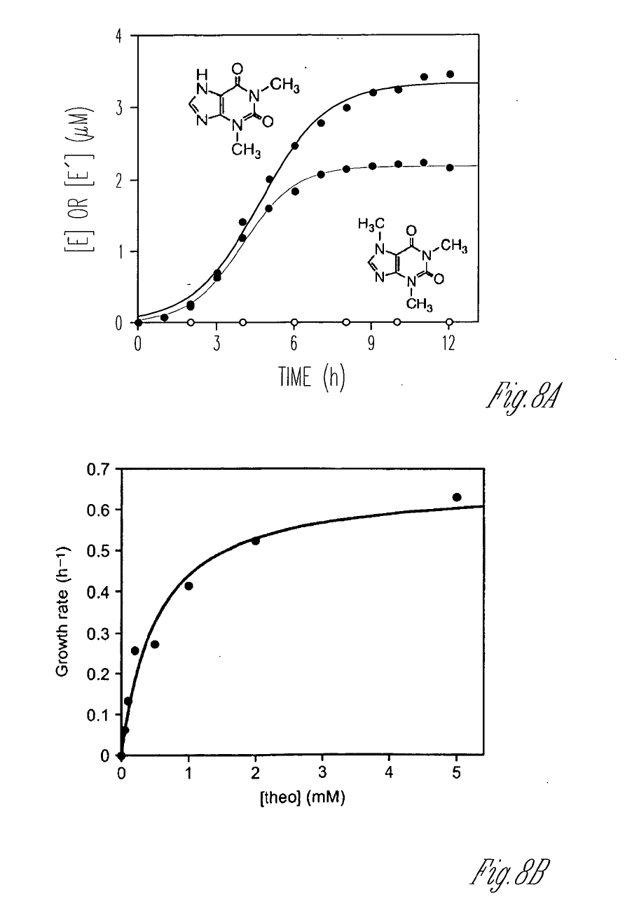

The maximum extents of synthesis of Etheo and E′theo were 3.3 and 2.2 μM, respectively. Exponential growth can be continued indefinitely, however, if a portion of the completed reaction mixture is transferred to a new mixture that contains a fresh supply of substrates. This is analogous to reseeding the PCR, but unlike the PCR remains dependent on the presence of the ligand throughout the amplification process, thus avoiding target-independent amplification. Following about a 100-fold amplification, 1% of the reaction mixture was transferred to a new reaction vessel that contained 5 μM each of the four substrates, but only those enzymes carried over in the transfer. Three successive incubations were carried out in this manner, resulting in 106-fold overall amplification after 15 hours (FIG. 9).

The exponential growth rate of cross-replicating aptazymes is dependent on the concentration of the corresponding ligand. This allows one to construct standardized curves that can be used to determine the concentration of ligand in an unknown sample. The theophylline-dependent aptazymes were exposed to theophylline levels ranging from 0.2 to 5.0 mM and the exponential growth rate of Etheo was determined. The growth rate as a function of theophylline concentration provided a saturation curve (FIG. 8B), which revealed that the aptazyme binds theophylline with a K_{d} of 0.51 mM. Thus, the aptazyme can be used to measure theophylline concentrations in the range of approximately 0.05-5 mM. The K_{d} for the theophylline aptamer in isolation is 0.1 μM (Jenison et al., 1994), indicating that the aptamer is significantly destabilized in the context of the aptazyme. No attempt was made to optimize the aptamer in this context, as has been done for other aptazymes using in vitro selection (Soukup and Breaker, 1999; Koizumi et al, 1999; Robertson and Ellington, 2000; Robertson and Ellington, 2001).

The FMN-dependent aptazymes also underwent exponential amplification in the presence, but not the absence, of their cognate ligand. The exponential growth rates of EFMN and E′FMN in the presence of 1 mM FMN were 0.58 and 0.70 hours⁻¹, respectively (FIG. 8C). The exponential growth rate of EFMN was determined in the presence of various concentrations of FMN, which provided a saturation curve (FIG. 8D) and revealed that the aptazyme binds FMN with a K_{d} of 0.068 mM. The same FMN aptamer has been linked to the hammerhead ribozyme and exhibited a K_{d} of 5 μM in that context (Robertson and Ellington, 2001). This compares with a K_{d} of 0.5 μM for the FMN aptamer in isolation (Robertson and Ellington, 2000).

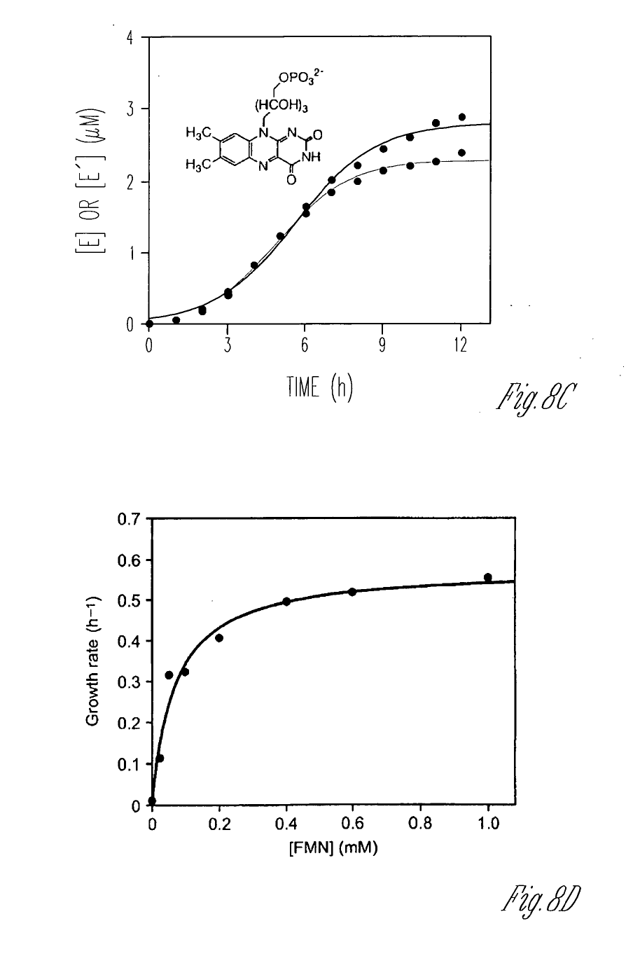

Ligand-dependent exponential amplification can be performed using a pair of cross-replicating aptazymes that recognize two different ligands. As an example, a reaction was carried out employing 0.02 μM each of Etheo and E′FMN, and 5 μM each of Atheo, A′FMN, B, and B′. There was no amplification in the absence of both ligands, and only linear amplification in the presence of either theophylline or FMN, but robust exponential amplification in the presence of both ligands (FIG. 10). This system can be regarded as performing a logical AND operation, providing exponential signal amplification that is dependent on the presence of two different inputs.

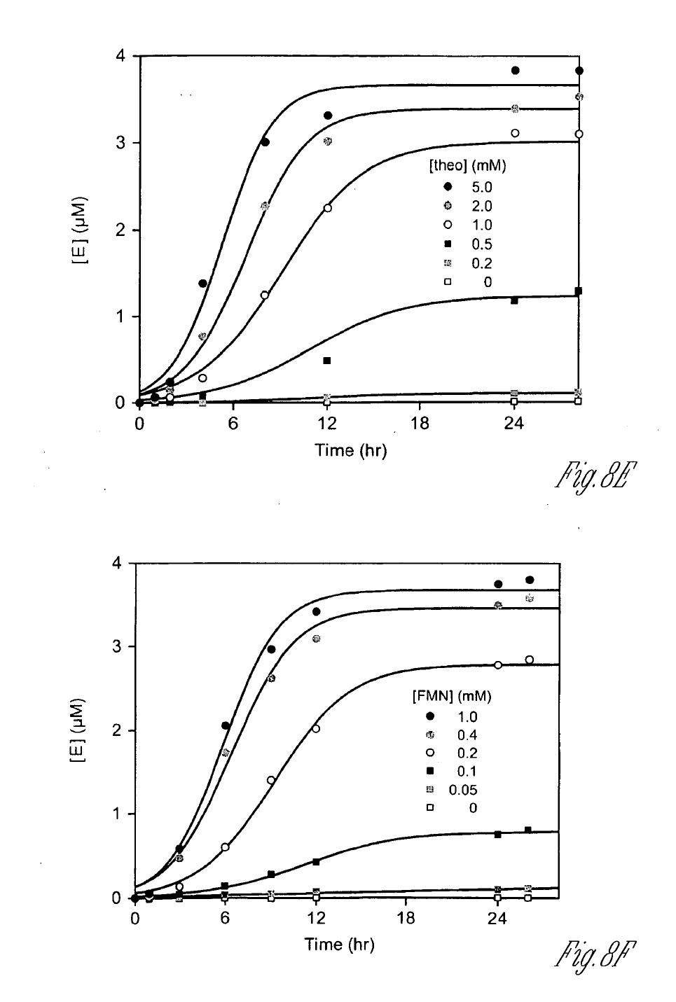

It is straightforward to carry out multiplexed ligand-dependent exponential amplification, employing two or more pairs of cross-replicating RNA enzymes that recognize their partners through distinct Watson-Crick pairing interactions (FIG. 6). Twelve pairs of cross-replicating RNA enzymes have been described in Example 1, one of which was chosen to contain the theophylline aptamer and another to contain the FMN aptamer (installing the same aptamer in both members of a cross-replicating pair). In the presence of either 5 mM theophylline or 0.7 mM FMN, only the corresponding RNA enzymes amplified exponentially, with growth rates for Etheo or EFMN of 0.35 or 0.43 hours⁻¹, respectively (FIG. 11). In the presence of both ligands, both pairs of cross-replicating enzymes amplified exponentially, with growth rates for Etheo and EFMN of 0.45 and 0.43 hours⁻¹, respectively.

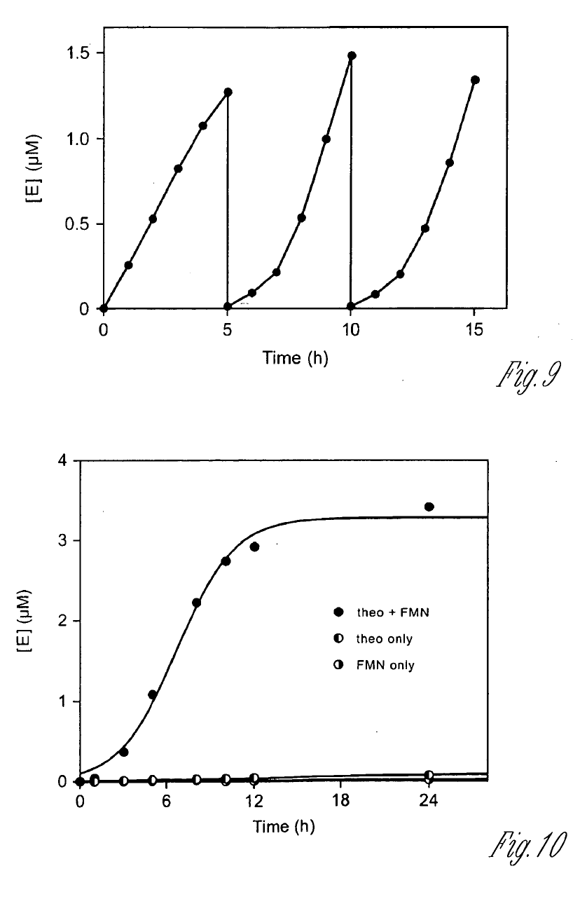

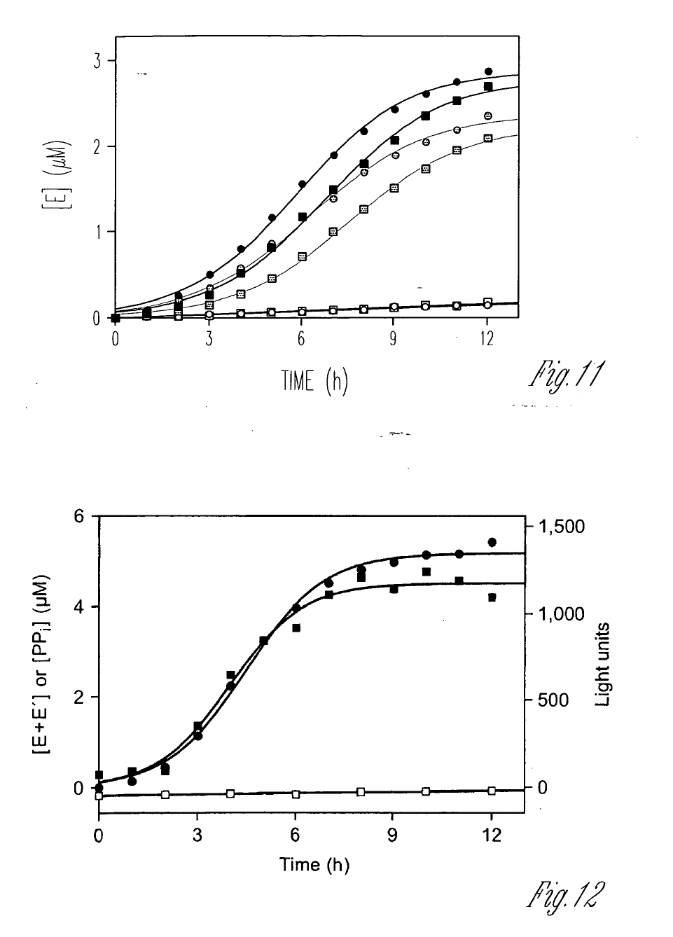

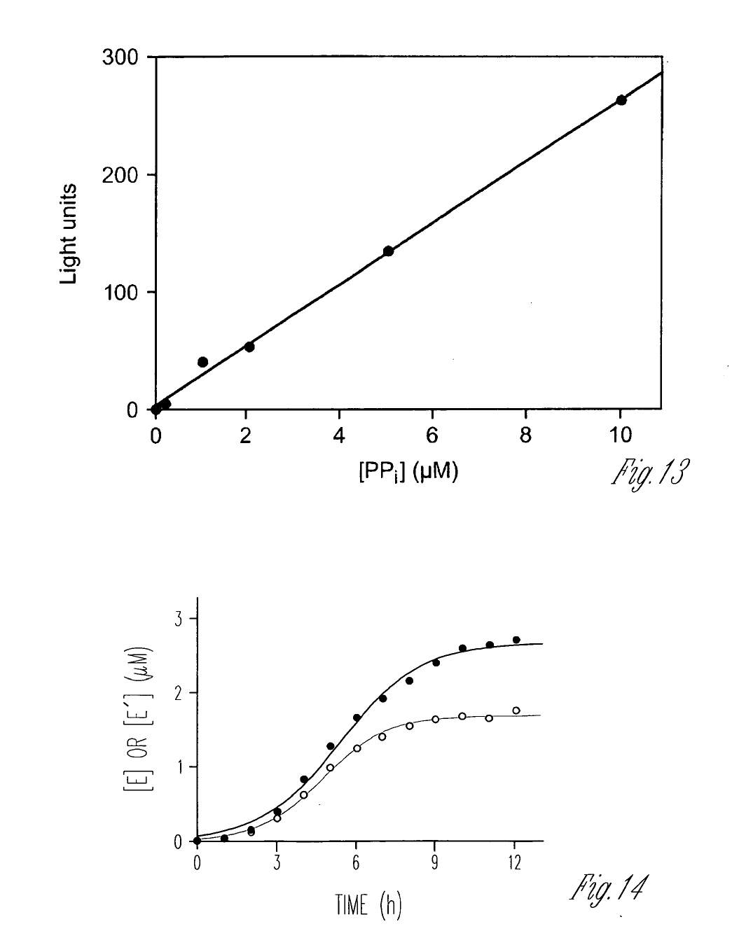

With each RNA-catalyzed ligation event, a 3′,5′-phosphodiester linkage is formed and one molecule of inorganic pyrophosphate is released (Rogers and Joyce, 2001). The released pyrophosphate can be used to generate a luminescent signal based on an ATP-regenerative luciferase assay (Ronaghi et al., 1996). A plot of light emission over the course of theophylline-dependent exponential amplification was nearly identical to that for formation of the ligated products (FIG. 12). The luminescent signal generated by various known concentrations of pyrophosphate was used to determine a conversion factor for relating light units to absolute concentrations of pyrophosphate (FIG. 13). These absolute concentrations were in close agreement with the absolute yield of ligated products over the course of exponential amplification (FIG. 12).

A limitation of autocatalytic aptazymes as a quantitative method for ligand-dependent exponential amplification is the need for the aptamer domain to bind its ligand with some requisite affinity, while remaining compatible with efficient cross-replication. The desired binding affinity usually is determined by the concentration of the ligand in its biological or environmental context. Methods are well established for generating RNA aptamers that bind a target protein or small molecule with a particular affinity (Fitzwater and Polisky, 1996; Ciesiolka et al., 1996). When these aptamers are placed in the context of an aptazyme, further optimization may be needed to regain the desired affinity. However, an additional problem arises when the concentration of substrates required for efficient exponential amplification (typically micromolar) exceeds the desired K_{d} for the aptamer-ligand interaction. One remedy would be to improve the Kₘ of the cross-replicating enzymes so that the enzyme-substrate interactions remain saturated even when the aptamer-ligand interaction is unsaturated. However, this approach would limit the amount of signal that could be generated for very low-abundance targets. Another approach, analogous to qPCR and other methods that link a rare recognition event to subsequent exponential amplification (Sano et al., 1992; Fredriksson et al., 2002) would be to employ RNA replication as a reporter that is triggered by a recognition event. Unlike qPCR such a process would be isothermal, but like qPCR it would not benefit from dynamic sensing of the ligand throughout the course of exponential amplification.

Another limitation of autocatalytic aptazymes is that the molecules are composed of RNA, which is susceptible to degradation by ribonucleases or inhibition by non-specific RNA-binding proteins. The theophylline-dependent aptazymes were rapidly degraded in the presence of 10% bovine calf serum, but were able to undergo unimpeded ligand-dependent exponential amplification in the presence of serum that had been deproteinized by phenol extraction (FIG. 14). Nuclease-resistant forms of the aptazymes may be needed, as has been done for most aptamers that are employed in a biological context (Lin et al., 1994; Green et al., 1995).

Aptamers and aptazymes have emerged as powerful tools for detecting and generating biochemical responses to a wide variety of ligands (Joyce, 2002; Orgel, 2004). Nature has exploited this mechanism in the operation of “riboswitches” (Winker et al., 2002; Mironov et al., 2002), which are ligand-dependent riboregulators that occur widely in biology (Winker et al., 2004; Mandal et al., 2004; Cheah et al., 2007). Scientists have engineered aptamers and aptazymes to sense proteins or small molecules (Nutia et al., 2003; Stojanonc et al., 2004; Kirby et al., 2004), to control gene expression (Werstuck and Green, 1998; Bayer and Smolke, 2005; Lynch et al., 2007), and to perform molecular computation (Yoshida and Yokobayashi, 2007; Win and Smolke, 2008). Autocatalytic aptazymes may be useful in some of these applications because they provide both specificity through dynamic sensing of the ligand and sensitivity due to ligand-dependent exponential amplification. Although several practical concerns still must be addressed, the ability to perform quantitative analysis of a variety of ligands under isothermal conditions is likely to have utility in medical diagnostics and environmental monitoring.

### REFERENCES

Barany, Proc. Natl. Acad. Sci. USA, 88:189 (1991).
Bayer and Smolke, Nat. Biotechnol., 23:337 (2005).
Bock et al., Proteomics, 4:609 (2004).
Brody & Gold, J. Biotechnol., 74:5 (2000).
Burgstaller and Famulok, Angew. Chemie, 33:1084 (1994).
Cadwell and Joyce, PCR Methods Applic., 2:28 (1992).
Cheah et al., Nature, 447:497 (2007).
Ciesiolka et al., Methods Enzymol., 267:315 (1996).
Crick, J. Mol. Biol., 38:367 (1968).
Engvall and Perlman, Immunochemistry, 8:871 (1971).
Fitzwater and Polisky, Methods Enzymol., 267:275 (1996).
Forster and Altman, Science, 249:783 (1990).
Fredriksson et al., Nat. Biotechnol., 20:473 (2002).
Hartig et al. Nat. Biotechnol., 20:717 (2002).
Hesselberth et al., Anal. Biochem., 312:106 (2003).
Jenison et al., Science, 263:1425 (1994).
Joyce, Nature, 418:214 (2002).
Kim and Joyce, Chem. Biol., 11:1505 (2004).
Kim et al., FEBS Lett., 582:2745 (2008).
Kirby et al., Anal. Chem., 76:4066 (2004).
Koizumi et al., Nature Struct. Biol., 6:1062 (1999).
Kossen et al., Chem. Biol., 11:807 (2004).
Lee et al., Nature, 390:591 (1997).
Levy and Ellington, Proc. Natl. Acad. Sci. USA, 100:6416 (2003)
Lin et al., Nucl. Acids Res., 22:5229 (1994).
Lynch et al., Chem. Biol., 14:173 (2007).
Mandal and Breaker, Nature Rev. Mol. Cell Biol., 5:451 (2004).
Mathews et al., J. Mol. Biol., 288:911 (1999).
Mironov et al., Cell, 111:747 (2002).
Morris et al., Proc. Natl. Acad. Sci. USA, 95:2902 (1998).
Ng et al., Nature Rev. Drug Discov., 5:123 (2006).
Nutiu and Li, J. Am. Chem. Soc., 125:4771 (2003).
Orgel, Crit. Rev. Biochem. Mol. Biol., 39:99 (2004).
Paul and Joyce, Proc. Natl. Acad. Sci. USA, 99:12733 (2002).
Pluthero, Nucleic Acids Res., 21:4850 (1993).
Pyle et al., Methods Enzymol., 317:140 (2000).
Rimmele, Chembiochem, 4:963 (2003).
Robertson and Ellington, Nature Biotechnol., 19:650 (2001).
Robertson and Ellington, Nucleic Acids Res., 28:1751 (2000).
Robertson et al., RNA, 10:114 (2004).
Rogers and Joyce, RNA, 7:395 (2001).
Ronaghi et al., Anal. Biochem., 242:84 (1996).
Sano et al., Science, 258:120 (1992).
Santoro and Joyce, Proc. Natl. Acad. Sci. USA, 94:4262 (1997).
Seetharaman et al., Nat. Biotechnol., 19:336 (2001).
Sievers and von Kiedrowski, Nature, 369:221 (1994).
Soukup and Breaker, Proc. Natl. Acad. Sci. USA, 96:3584 (1999).
Stojanovic and Kolpashchikov, J. Am. Chem. Soc., 126:9266 (2004).
Szostak et al., Nature, 409:387 (2001).
Tang and Breaker, Chem. Biol., 4:453 (1997).
Tucker and Breaker, Curr. Opin. Struct. Biol., 15:342 (2005).
Vaish et al., Nature Biotechnol., 20:810 (2002).
Vaish et al., RNA, 9:1058 (2003).
Vartanian et al., Nucleic Acids Res., 24:2627 (1996).
Werstuck and Green, Science, 282:296 (1998).
Win and Smolke, Science, 322:456 (2008).
Winkler et al., Nature, 419:952 (2002).
Winkler et al., Nature, 428:281 (2004).
Wu and Wallace, Genomics, 4:560 (1989).
Yoshida and Yokobayashi, Chem. Commun., 2007:195 (2007).
Zuker, Nucleic Acids Res., 31:3406 (2003)

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to detect a selected molecule in a sample, comprising:
a) contacting a sample suspected of having the selected molecule, a pair of cross-catalytic nucleic acid molecules, wherein at least one of the pair comprises a ligand binding domain for the selected molecule, and substrates for each of the pair, under conditions that result in selected molecule-dependent self-replication of at least one of the pair and exponential amplification of at least one of the pair that is dependent on the self-replication and optionally dependent on the selected molecule; and
b) detecting or determining the presence or amount of the amplified nucleic acid molecule, thereby detecting or determining the presence or amount of the selected molecule in the sample.

2. The method of claim 1 wherein the Kd of the selected molecule and ligand binding domain is about 1 to about 100 nM.

3. The method of claim 1 or 2 wherein the amplification is under isothermal conditions.

4. The method claim 1 wherein the pair of cross-catalytic molecules and/or the substrates therefore are nuclease resistant.

5. The method of claim 1 wherein the selected molecule is a soluble molecule present in physiologic fluid.

6. The method of claim 1 wherein the sample is a blood sample, serum sample, plasma sample, urine sample, or spinal fluid sample.

7. The method of claim 1 wherein the selected molecule is a peptide or protein.

8. The method of claim 1 wherein the selected molecule is a drug.

9. The method of claim 1 wherein the selected molecule is a cellular molecule.

10. The method of claim 1 wherein concentrations of about 1 to 100 μM of the selected molecule in the sample are detected or determined.

11. The method of claim 1 wherein each nucleic acid molecule of the pair comprises a ligand binding domain.

12. The method of claim 11 wherein the ligand binding domains bind the same molecule.

13. The method of claim 11 wherein the ligand binding domains bind different molecules.

14. The method of claim 1 wherein only one of the pair of nucleic acid molecules comprises the ligand binding domain.

15. The method of claim 1 wherein the doubling time is less than about 1 hour.

16. A composition comprising a pair of cross-replicating nucleic acid enzyme molecules that are capable of exponential amplification in the presence of substrates therefor and in the absence of thermal cycling and proteins or other biological molecules.

17. The composition of claim 16 wherein one of the enzymes comprises a ligand binding domain.

18. The composition of claim 16 wherein both of the enzymes comprise a ligand binding domain.

19. An isolated cross-replicating nucleic acid molecule comprising a catalytic domain and a ligand binding domain.

20. The isolated nucleic acid molecule of claim 19 which is an RNA molecule.

21. The isolated nucleic acid molecule of claim 19 or 20 which is a ligase.

22. The isolated nucleic acid molecule of claim 21 wherein the ligand binding domain binds a metabolite, a protein, a drug, s toxin or a cell

23-28. (canceled)
